SEQUENCE LISTING (12 sequences)

SEQ ID NO: 1 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 2 - protein, 352 aa, Mus musculus
MAPLGYLLVLCSLKQALGSYPIWWSLAVGPQYSSLSTQPILCASIPGLVPKQLRFCRNYVEIMPSVAEGVKAGIQECQHQFRGRRWNCTTVSNSLAIFGPVLDKATRESAFVHAIASAGVAFAVTRSCAEGSAAICGCSSRLQGSPGEGWKWGGCSEDIEFGGMVSREFADARENRPDARSAMNRHNNEAGRQAIASHMHLKCKCHGLSGSCEVKTCWWSQPDFRTIGDFLKDKYDSASEMVVEKHRESRGWVETLRPRYTYFKVPTERDLVYYEASPNFCEPNPETGSFGTRDRTCNVSSHGIDGCDLLCCGRGHNARTERRREKCHCVFHWCCYVSCQECTRVYDVHTCK

SEQ ID NO: 3 - protein, 352 aa, Xenopus laevis
MGCFGYLLLIIGLHQVLATYPIWWSLAVGQQYSSLGTQPIPCGTIPGLVAKQMRFCRNYMEIMPSVAEGVKIGIQECQHQFRGRRWNCTTVNDNLAIFGPVLDKATRESAFVHAIASAGVAFAVTRSCAEGSATICGCDTHHKGPPGEGWKWGGCSEDMDFGSMVSREFADARENRPDARSAMNRHNNEAGRTSILDHRHLKCKCHGLSGSCEVKTCWWSQPDFRVIGDYLKDKYDSASEMVVEKHRESRGWVETLRPKYTFFKPPIERDLIYYESSPNFCEPNPETGSFGTRDRECNVTSHGIDGCDLLCCGRGQNTRTEKRKEKCHCIFHWCCYVSCQECMRVYDVHTCK

SEQ ID NO: 4 - protein, 352 aa, Gallus gallus
MASFGYFLFLCGLSQALSSYPIWWSLAIGHQYSSLGTQPILCGSIPGLVPKQLRFCRNYVEIMPSVAEGVKIGIQECQHQFRGRRWNCTTVNDSLAIFGPVLDKATRESAFVHAIASAGVAFAVTRSCAEGSATICGCDTRHKGSPGEGWKWGGCSEDVEFGSMVSREFADARENRPDARSAMNRHNNEAGRTSIIELMHLKCKCHGLSGSCEVKTCWWSQPDFRVIGDYLKDKYDSASEMVVEKHRESRGWVETLRPKYNFFKAPTEKDLVYYENSPNFCEPNPETGSFGTRDRICNVTSHGIDGCDLLCCGRGHNTRTEKRKEKCHCIFHWCCYVRCQECIRVYDVHTCK

SEQ ID NO: 5 - DNA, 2346 bp, Homo sapiens
atggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaa

SEQ ID NO: 6 - DNA, 3565 bp, Mus musculus
gcggcgccatcttaagccctcgctcggtggcggccgcgtcagctcgtgtcctgtgaagcccgcggcccggggaggcggagacggagcacggtgggcgccgagccgtcagtgcaggaggccgaggccgagcgggcggccgcgagtgagcagcgcgcgggcctgagggtacctgaagctcagcgcacagctgctgtgacaccgctgcgtggacaatggctactcaagctgacctgatggagttggacatggccatggagccggacagaaaagctgctgtcagccactggcagcagcagtcttacttggattctggaatccattctggtgccaccaccacagctccttccctgagtggcaagggcaaccctgaggaagaagatgttgacacctcccaagtcctttatgaatgggagcaaggcttttcccagtccttcacgcaagagcaagtagctgatattgacgggcagtatgcaatgactagggctcagagggtccgagctgccatgttccctgagacgctagatgagggcatgcagatcccatccacgcagtttgacgctgctcatcccactaatgtccagcgcttggctgaaccatcacagatgttgaaacatgcagttgtcaatttgattaactatcaggatgacgcggaacttgccacacgtgcaattcctgagctgacaaaactgctaaacgatgaggaccaggtggtagttaataaagctgctgttatggtccatcagctttccaaaaaggaagcttccagacatgccatcatgcgctcccctcagatggtgtctgccattgtacgcaccatgcagaatacaaatgatgtagagacagctcgttgtactgctgggactctgcacaacctttctcaccaccgcgagggcttgctggccatctttaagtctggtggcatcccagcgctggtgaaaatgcttgggtcaccagtggattctgtactgttctacgccatcacgacactgcataatctcctgctccatcaggaaggagctaaaatggcagtgcgcctagctggtggactgcagaaaatggttgctttgctcaacaaaacaaacgtgaaattcttggctattacaacagactgccttcagatcttagcttatggcaatcaagagagcaagctcatcattctggccagtggtggaccccaagccttagtaaacataatgaggacctacacttatgagaagcttctgtggaccacaagcagagtgctgaaggtgctgtctgtctgctctagcaacaagccggccattgtagaagctggtgggatgcaggcactggggcttcatctgacagacccaagtcagcgacttgttcaaaactgtctttggactctcagaaacctttcagatgcagcgactaagcaggaagggatggaaggcctccttgggactctagtgcagcttctgggttccgatgatataaatgtggtcacctgtgcagctggaattctctctaacctcacttgcaataattacaaaaacaagatgatggtgtgccaagtgggtggcatagaggctcttgtacgcaccgtccttcgtgctggtgacagggaagacatcactgagcctgccatctgtgctcttcgtcatctgaccagccggcatcaggaagccgagatggcccagaatgccgttcgccttcattatggactgcctgttgtggttaaactcctgcacccaccatcccactggcctctgataaaggcaactgttggattgattcgaaaccttgccctttgcccagcaaatcatgcgcctttgcgggaacagggtgctattccacgactagttcagctgcttgtacgagcacatcaggacacccaacggcgcacctccatgggtggaacgcagcagcagtttgtggagggcgtgcgcatggaggagatagtagaagggtgtactggagctctccacatccttgctcgggacgttcacaaccggattgtaatccgaggactcaataccattccattgtttgtgcagttgctttattctcccattgaaaatatccaaagagtagctgcaggggtcctctgtgaacttgctcaggacaaggaggctgcagaggccattgaagctgagggagccacagctcccctgacagagttactccactccaggaatgaaggcgtggcaacatacgcagctgctgtcctattccgaatgtctgaggacaagccacaggattacaagaagcggctttcagtcgagctgaccagttccctcttcaggacagagccaatggcttggaatgagactgcagatcttggactggacattggtgcccagggagaagcccttggatatcgccaggatgatcccagctaccgttcttttcactctggtggatacggccaggatgccttggggatggaccctatgatggagcatgagatgggtggccaccaccctggtgctgactatccagttgatgggctgcctgatctgggacacgcccaggacctcatggatgggctgcccccaggtgatagcaatcagctggcctggtttgatactgacctgtaaatcgtcctttaggtaagaaagcttataaaagccagtgtgggtgaatactttactctgcctgcagaactccagaaagacttggtagggtgggaatggttttaggcctgtttgtaaatctgccaccaaacagatacataccttggaaggagatgttcatgtgtggaagtttctcacgttgatgtttttgccacagcttttgcagcgttatactcagatgagtaacatttgctgttttcaacattaatagcagcctttctctctatacagctgtagtgtctgaacgtgcattgtgattggcctgtagagttgctgagagggctcgaggggtgggctggtatctcagaaagtgcctgacacactaaccaagctgagtttcctatgggaacagtcgaagtacgctttttgttctggtcctttttggtcgaggagtaacaatacaaatggatttggggagtgactcacgcagtgaagaatgcacacgaatggatcacaagatggcgttatcaaaccctagccttgcttgttctttgttttaatatctgtagtggtgctgactttgcttgcttttattttttgcagtaactgttagtttttaagtagtgttatgttctagtgaacctgctacagcaatttctgatttctaagaaccgagtaatggtgtagaacactaattcataatcacgctaattgtaatctggagacgtgtaacattgtgtagccttttgtataaatagacagatagaaatggtccgattagtttcctttttaatatgcttaaaataagcaggtggatctatttcatgtttttgaacaaaaactttatcggggatacgtgcggtagggtaaatcagtaagaggtgttatttgagccttgttttggacagtataccagttgccttttatcccaaagttgttgtaacctgctgtgatacaatgcttcaacagatgcggttatagaaatggttcagaattaaacttttaattcattcaaaaaaaaaaaaaaaaaa

SEQ ID NO: 7 - DNA, 3281 bp, Gallus gallus
ggcacgagagcagcagcagccggagcccggggggacgctgcgctctcgcctccgtgccacagtctctgaaaggtagcaggaaagagttctgggagcacagcaaggaacatggcaacccaagctgacttgatggagttggatatggccatggagccagacagaaaagctgcagtcagtcattggcagcagcagtcatatctggactctggtatccattccggtgccacgacaactgctccctctttgagtggcaaaggaaatcctgaagaggaagatgtggacacaacgcaagtcctgtatgagtgggagcaagggttctctcagtcctttacccaggagcaagttgctgatattgatggccaatatgcaatgactagagctcagagagtgctttctgctatgttccccgaaacactggatgaaggaatgcaaatcccatccacacaatttgatgctgcccatccaactaatgtgcagcgcctggctgagccatcccagatgctaaaacatgctgttgttaatttgataaactatcaggatgatgctgaacttgcaactcgtgcaatcccagaactgaccaaactgttgaatgatgaggaccaggtggtggtaaacaaggctgcggttatggttcatcagctatccaaaaaggaagcatctcgccatgctattatgaggtctcctcaaatggtatctgcaattgtgcgtaccatgcaaaatacaaacgatgtggaaacagcccgctgtactgcaggcacactacacaatctctcacatcaccgtgaaggcttgttggcaatcttcaaatcaggaggcatccctgcgttggttaaaatgcttgggtctccggtggattctgtgttgttctatgccattactactcttcacaatctcctgttacatcaggagggagccaaaatggctgtccgtctggctggagggctgcaaaaaatggttgccctgctcaacaagacaaatgtgaaattcttggccatcacgacagactgtcttcagattttagcctatggcaatcaagaaagtaagctgattattctggcaagcggtggaccccaagctctagtaaacataatgaggacctacacttatgagaaactattgtggaccacaagtagggtgctgaaggtgttgtcagtctgctccagcaacaaacctgctattgttgaggctggtgggatgcaagctttaggactccaccttacagatccaagccagcgtcttgtccagaactgtctctggaccctgagaaatttgtcagatgcagcaaccaagcaggagggaatggaaggccttctaggaactcttgttcagcttttaggatcagatgacattaatgttgtgacttgcgctgccggaatcctttctaaccttacttgcaacaattacaagaacaagatgatggtctgccaggttggtggcatcgaggctcttgtgcgcacagttcttcgggctggagacagggaagacatcacagaacctgctatttgtgcgctccgtcacctcaccagcagacatcaggaagctgaaatggctcaaaatgcagtacgtctccattacggactcccagtggtggttaaactgttgcacccaccttcacactggcctttgatcaaggctactgttgggttgattcgcaatctcgcgctctgccctgcaaaccatgccccactgcgtgaacaaggtgctatcccacggctagttcagctgctggttagagcacatcaagatacccagcgacgtacttccatgggtggaacgcaacagcagtttgtggagggtgtgcgcatggaggaaatcgttgagggctgtactggagccctgcatattcttgcacgtgatgttcacaatcgaattgtaatcaggggtctaaataccattccactatttgtgcagttgttgtactcccccattgagaatatccagagagtagctgcgggtgtactttgtgaacttgctcaagacaaggaagcagctgaagcaattgaagctgaaggcggaactgcccctttaacagaactgcttcattccaggaatgagggtgttgcaacatatgcagctgcagtgctgttcagaatgtcggaggacaaaccacaagactacaagaagcgactttcagttgaactgacaagctctctgttccggactgagccaatggcttggaacgagacagcggatcttggacttgacattggtgcccagggagaacctcttggataccgcccagatgatcctagctaccgttctttccactctggcggatacggtcaggatgccttgggtatggaccctatgatggaacatgaaatgggtggccaccaccctggtgctgactacccagttgatggtctgccagatcttggccatgcccaggaccttatggatgggctgcctccaggtgacagtaatcagttggcctggttcgatactgacctgtaaatcatcctttagctgtatcatctgaatgaacttgcattgattggcctgtagagttgctgagagggctcgaggggtgggctagtatctcagaaagtgcctgacacactaaccaagctgagtttcctatgggaacaattgaagtaaactttttgttctggtcctttttggtcgaggagtaataatacaaatggattttgggagtgattcaagaaacgaggaatgcacaagaatgaattgcaagatggaatttatcaaaccctagccttgcttgttaaaaatttattattttttttaaatctctgtaatggtactgacctttgcttgctttgaaagtagcctttcttttcgcagtaattgttgttagggttttttttttaagtctctcgtagtattaagttatagtgaatatgctacagccgtttctaatttttaaggattgagtaaaggtgtagaacactaattcataatcgctctaactgtattctgaataaagtgtaacattgtgtagcctttttgtataaaaaaaactagacaaatagaaatggtccaattagtttcctttttaatatgcttaaaataagcaggtggatctatttcatgtttttgatcaaaaactttatctggatatgtctgggtaggggccagtaagaactgtttatttggaaccttgtattggacagtttaccagttgccttttatcccaaagttattgtagcctgctgtgatacagatgcttcatgagaaaaatgcagttataaaatggttcaaaattaaagtaaacttttaattcaaaaaaaaaaaaaaaaaactcgagagtacttctagag

SEQ ID NO: 8 - DNA, 3385 bp, Xenopus laevis
gtggtgtgcatttgttcatggcttgcatctgaaaggggtcacaaataaaagcggggaaaagtaaatcaacgctcttgccaccacaccccgtccgcctttaggtgggacatcattcgggagctgcaatatctggaggcggtgtaggagacggccagtaccgggcgaaacacggtgacggccggaaccgggggacatcgagccgacggcttgtaatccgcaacctggttctttggaaacggttgaaaaatggcaactcaagcagatctaatggagctggacatggccatggagccagaccgaaaggcagcagtgagccactggcagcagcagtcttacctggattctgggattcattctggagcaaccaccacagcaccatctttgagtggcaaaggaaacccagaggatgaagatgtggataccaaccaagttttgtatgagtgggagcagggcttctctcagtccttcactcaagatcaagtggctgatattgatggccagtatgccatgacaagagcccagcgagttcgtgctgcaatgtttccagaaacccttgatgaaggcatgcagattccatccacacaatttgactctgcacacccaacaaatgtgcaacgtttagcagagccttcccagatgctcaaacacgctgtggtcaacttgatcaattaccaggatgatgctgaattggccactcgagcaatccccgagctgacgaaactgcttaatgacgaggaccaggttgtagttaacaaggctgctgtaatggttcaccagctgtcaaagaaggaagcctcacgccatgctataatgcgctcaccacagatggtgtctgctatagttcgtacaatgcagaacacaaatgatgtagagactgcacgatgcactgcgggaacacttcacaacctttctcaccatcgtgaaggcttgttggccatatttaaatctggaggcattcctgctctagttaaaatgcttggctcaccagttgactctgtactattctatgccatcacaacactgcacaatctacttctccatcaagaaggagcaaaaatggctgtacgtctggctggtggcttgcagaaaatggttgcccttctcaataaaactaatgtcaagtttttggcaattacaacagactgtcttcaaatattggcctatggaaatcaagaaagcaagttgataattcttgcaagtgggggaccacaagcgttggtaaatatcatgaggacttacagttatgagaaacttctgtggaccacaagtcgggtgcttaaggtgctatccgtgtgctctagcaacaagcctgctatagttgaagctggtggaatgcaagctttaggactccatctcacagactcaagccaacgtttggttcagaattgtctttggacactaagaaacctttcagatgcagcaactaaacaggagggaatggaaggtcttcttgggaccctggttcagcttcttggatcagatgacattaatgttgtaacttgtgcagctggtatattgtccaacctcacctgcaacaactacaaaaacaaaatgatggtttgtcaagttggaggcattgaagcacttgttcgcacagttctacgtgctggggatagagaagacattactgaacctgcaatttgtgctctgcgtcaccttaccagcaggcaccaggaagcagagatggctcaaaatgcagtacgtctccattatggacttccagtggtggtgaaactcttgcacccgccttcacactggcctttgataaaggctactgttggcttgatccgcaatcttgccctttgtccagccaatcatgctccactgcgtgaacagggtgccattccccgacttgttcagttgcttgttcgtgcacaccaggacacacagaggcgcacttcaatgggtggaacacaacagcagtttgtggagggtgtccgcatggaggagattgttgaaggctgcacaggtgctcttcacattttggctcgtgacatccacaaccgaattgtaatcagaggtcttaacaccattccattatttgtgcagttgctgtactctcctattgaaaatatccaaagagtggcagcaggtgtgctgtgtgagctggcgcaagacaaagaggctgctgaagctattgaagctgaaggtgcaactgctcctcttactgagctgcttcactctagaaatgaaggtgttgcaacttatgcagctgctgttctgttccgtatgtctgaggacaaaccccaggactacaagaagcgtctgtcagttgagttgaccagctctctcttcaggaccgagccaatgccatggaatgaggctgcagaccttggtcttgatattggtgcccaaggcgaacctcttggctacagacaggatgattcaagctaccgatctttccatgctgctggctatggtcaggatgcaatgggtatggactccatgatggatcatgacatgggaggtcaccacccaggggcagactatccagttgatggacttcctgatttgagtcatgcccaagatcttatggatggactacctccaggtgatagcaaccaactggcctggtttgacactgacttgtaaatatctacttttttgttattgtccagatgaatctgcattgtgattggcctgtagtttgctgagagggcaggaggggggctggtaatctcagaaagtgcctgacacactaaccaagctgagtttcctatgggaacaacggaagaaaactttgttctggtcctttatttggtcagaggaacaaaaaccaatggattttggaagtgactcacatcaagaatgcacaagaatagttttgcaagctggaactgatcagaaacactagccttgcttattttaaattttattttttattttttttcctttttttatctgtatggtaccaacctcgcttgctttctttagtatccagatttcttttcctttctaaggtaacccaagtctctgtagcgttcagttagtgcataatgctaaggcagttttctcatttttaaggatcaagtgtagaacactaaatcataatcacttattgtatcctgaagtggaacattgtgtagcttttttttttttttttttttttctttttgtataaaaatagaaatggtccaaatagtttttctttttaatatgctcagtctgatcattttttgatcaaagcttttatctggaatgtctgggaaggggccagtaagaatctcgacttatttggaacctgtaatggacattttaccagttgccttttatcccaaagttattgtactgaagccacagatgcttaatgaaaatcacattataatatagctccgaagaattaaagtggacttttaatgcaaaaaaaaaaaaaaaaaaaaaaaaa

SEQ ID NO: 9 - protein, 721 aa, Homo sapiens
QVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 10 - protein, 781 aa, Mus musculus
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEALGYRQDDPSYRSFHSGGYGQDALGMDPMMEHEMGGHHPGADYPVDGLPDLGHAQDLMDGLPPGDSNQLAWFDTDL

SEQ ID NO: 11 - protein, 781 aa, Gallus gallus
MATQADLMELDMAMEPDRKAAVSHWQQQSYLDSGIHSGATTTAPSLSGKGNPEEEDVDTTQVLYEWEQGFSQSFTQEQVADIDGQYAMTRAQRVLSAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGGTAPLTELLHSRNEGVATYAAAVLFRMSEDKPQDYKKRLSVELTSSLFRTEPMAWNETADLGLDIGAQGEPLGYRPDDPSYRSFHSGGYGQDALGMDPMMEHEMGGHHPGADYPVDGLPDLGHAQDLMDGLPPGDSNQLAWFDTDL

SEQ ID NO: 12 - protein, 781 aa, Xenopus laevis
MATQADLMELDMAMEPDRKAAVSHWQQQSYLDSGIHSGATTTAPSLSGKGNPEDEDVDTNQVLYEWEQGFSQSFTQDQVADIDGQYAMTRAQRVRAAMFPETLDEGMQIPSTQFDSAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYSYEKLLWTTSRVLKVLSVCSSNKPAIVEAGGMQALGLHLTDSSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDIHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMPWNEAADLGLDIGAQGEPLGYRQDDSSYRSFHAAGYGQDAMGMDSMMDHDMGGHHPGADYPVDGLPDLSHAQDLMDGLPPGDSNQLAWFDTDL

(19) United States
(12) Patent Application Publication
(10) Pub. No.: US 2016/0194608 A1
(43) Pub. Date: Jul. 7, 2016

(54) Methods for Inducing Cardiomyogenesis

(71) Applicant: THE J. DAVID GLADSTONE INSTITUTES, San Francisco, CA (US)

(72) Inventors: Deepak Srivastava, San Francisco, CA (US); Chulan Kwon, Ellicot City, MD (US)

(21) Appl. No.: 14/814,452

(22) Filed: Jul. 30, 2015

Related U.S. Application Data
(62) Division of application No. 12/513,657, filed on Dec. 14, 2009, now abandoned.
(60) Provisional application No. 60/858,145, filed on Nov. 9, 2006.

(51) Int. Cl. C12N 5/077

(52) U.S. Cl. CPC C12N 5/0657; C12N 2501/415

(57) ABSTRACT

The present invention provides methods of inducing cardiomyogenesis and expansion of cardiac progenitors in a population of stem cells or progenitor cells, the methods generally involving inducing a canonical Wnt signaling pathway in the stem cells or progenitor cells. The present invention provides methods of generating a population of cardiomyocytes or cardiac progenitors from a population of stem cells or progenitor cells, the methods generally involving contacting the stem cells or progenitor cells with an agent that induces canonical Wnt signaling. A subject method is useful for generating a population of cardiomyocytes or cardiac progenitors, which can be used in research and therapeutic applications.

### BACKGROUND

There is a need in the art for methods of generating cardiomyocytes, for research applications, as well as for therapeutic applications.

### SUMMARY OF THE INVENTION

The present invention provides methods of inducing cardiomyogenesis and expansion of cardiac progenitors in a population of stem cells or progenitor cells, the methods generally involving inducing a canonical Wnt signaling pathway in the stem cells or progenitor cells. The present invention provides methods of generating a population of cardiomyocytes or cardiac progenitors from a population of stem cells or progenitor cells, the methods generally involving contacting the stem cells or progenitor cells with an agent that induces canonical Wnt signaling. A subject method is useful for generating a population of cardiomyocytes or cardiac progenitors, which can be used in research and therapeutic applications.

### BRIEF DESCRIPTION OF THE DRAWINGS

FIGS. 1A and 1B. FIG. 1A depicts generation of tissue-specific null and stable β-catenin. FIG. 1B depicts Western blot of ventricles from Nkx2.5-cre, ctnnb1^{tm2Kem} heterozygous, homozygous (left) and wildtype, Nkx2.5-cre, β-catenin/loxP(ex3) heterozygous (right) embryos.

FIG. 2 depicts expression profiles of Brachyury.

FIGS. 3A and 3B depict expression profiles of early and late cardiac genes.

FIGS. 4A-E depict the effect of canonical Wnt signaling on cardiac induction and differentiation in ES cells.

FIG. 5 depicts histograms showing YFP+ cell populations from control (Islet1-cre, left), wildtype (Rosa-YFP; Islet1-cre, middle) and mutant (Islet1-cre; β-catenin(ex3)loxP, right) embryos at E9.5.

FIG. 6 depicts quantitative real-time RT-PCR of indicated genes from YFP+ cells sorted from WT (Rosa-YFP; Islet1-cre, left bars) and Mut (Rosa-YFP; Islet1-cre; β-catenin(ex3)loxP, right bars) embryos.

FIG. 7 depicts the effect of canonical Wnt on human ES cells.

FIGS. 8A and 8B provide an alignment of amino acid sequences of Wnt3a proteins.

FIGS. 9A-H provide an alignment of nucleotide sequences encoding β-catenin.

FIGS. 10A-C provide an alignment of amino acid sequences of β-catenin.

### DEFINITIONS

As used herein, the term “stem cell” refers to an undifferentiated cell that can be induced to proliferate. The stem cell is capable of self-maintenance, meaning that with each cell division, one daughter cell will also be a stem cell. Stem cells can be obtained from embryonic, post-natal, juvenile or adult tissue. The term “progenitor cell,” as used herein, refers to an undifferentiated cell derived from a stem cell, and is not itself a stem cell. Some progenitor cells can produce progeny that are capable of differentiating into more than one cell type.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” means the amount of a compound or a number of cells that, when administered to a mammal or other subject for treating a disease, is sufficient to effect such treatment for the disease. The “therapeutically effective amount” will vary depending on the compound or the cell, the disease and its severity and the age, weight, etc., of the subject to be treated.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a stem cell” includes a plurality of such stem cells and reference to “the cardiomyocyte” includes reference to one or more cardiomyocyte and equivalents thereof known to those skilled in the art, and so forth. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides methods of inducing cardiomyogenesis and expansion of cardiac progenitors in a population of stem cells or progenitor cells, the methods generally involving inducing a canonical Wnt signaling pathway in the stem cells or progenitor cells. The present invention provides methods of generating a population of cardiomyocytes or cardiac progenitors from a population of stem cells or progenitor cells, the methods generally involving contacting the stem cells or progenitor cells with an agent that induces canonical Wnt signaling. A subject method is useful for generating a population of cardiomyocytes or cardiac progenitors, which can be used in research and therapeutic applications.

### Methods of Inducing Cardiomyogenesis and Expansion of Cardiac Progenitors

The present invention provides methods of inducing cardiomyogenesis in a population of stem cells or progenitor cells; and methods for expansion of (increasing the numbers of) cardiac progenitors. In some embodiments, the methods generally involving inducing a canonical Wnt signaling pathway in the stem cells or progenitor cells. In other embodiments, the method comprising increasing the level of β-catenin in the stem cells or progenitor cells.

In some embodiments, using a subject method, at least about 10% of the stem cell or progenitor cell population differentiates into cardiomyocytes. For example, in some embodiments, from about 10% to about 50% of the stem cell or progenitor cell population differentiates into cardiomyocytes. In other embodiments, at least about 50% of the stem cell or progenitor cell population differentiates into cardiomyocytes. For example, in some embodiments, from about 50% to about 60%, from about 60% to about 70%, from about 70% to about 80%, or from about 80% to about 90%, or more, of the stem cell or progenitor cell population differentiates into cardiomyocytes.

In some embodiments, a subject method provides for an increase in the number of cardiac progenitors. For example, in some embodiments, a subject method provides for inducing proliferation in cardiac progenitors, thereby increasing the number of cardiac progenitors. Thus, e.g., in some embodiments, a subject method results in an increase of at least about 25%, at least about 50%, at least about 100% (or two-fold), at least about 5-fold, at least about 10-fold, at least about 25-fold, at least about 50-fold, or at least about 100-fold, or more, in the number of cardiac progenitors.

In some embodiments, a subject method provides for an increase in the number of beating embryoid bodies from a population of stem cells or progenitor cells, e.g., a subject method can provide for an increase in the number of beating embryoid bodies of from about 10% to about 50%, from about 50% to about 100% (or 2-fold), from about 2-fold to about 5-fold, from about 5-fold to about 10-fold, from about 10-fold to about 25-fold, from about 25-fold to about 50-fold, or from about 50-fold to about 100-fold, or greater than 100-fold.

In some embodiments, a subject method results in an increase in the number of cells in a population that express one or more of Isl1 (Islet 1), tropomyosin, Nkx2.5, Tbx5, Hand2, and a sarcomeric gene. In some embodiments, a subject method results in an increase of from about 10% to about 50%, from about 50% to about 100% (or 2-fold), from about 2-fold to about 5-fold, from about 5-fold to about 10-fold, from about 10-fold to about 25-fold, from about 25-fold to about 50-fold, or from about 50-fold to about 100-fold, or greater than 100-fold in the number of cells in a population that express one or more of Isl1 (Islet 1), tropomyosin, Nkx2.5, Tbx5, Hand2, and a sarcomeric gene.

Whether a stem cell or progenitor cell has differentiated into a cardiomyocyte can be readily determined. For example, in some embodiments, differentiation into a cardiomyocyte is ascertained by detecting cardiomyocyte-specific markers produced by the cell. Suitable cardiomyocyte-specific cell surface markers include, but are not limited to, troponin and tropomyosin.

In some embodiments, a subject method is carried out in vitro. In other embodiments, a subject method is carried out in vivo. Where a subject method is carried out in vitro, in some embodiments, the cardiomyocytes are subsequently introduced into a living organism.

In some embodiments, a subject method is carried out wherein the stem cells or progenitor cells are present in a matrix. In some of these embodiments, a subject method is suitable for producing an artificial heart tissue.

### Inducing a Canonical Wnt Signaling Pathway

The present invention provides methods of inducing cardiomyogenesis and/or expansion of cardiac progenitors in a population of stem cells or progenitor cells. In some embodiments, the methods involve inducing a canonical Wnt signaling pathway in the stem cells or progenitor cells. The canonical Wnt pathway describes a series of events that occur when Wnt ligands bind to cell-surface receptors of the Frizzled family, causing the receptors to activate Dishevelled family proteins and ultimately resulting in a change in the amount of β-catenin that reaches the nucleus. Smalley et al. ((2005) J. Cell Sci. 118:5279); Logan and Nusse (2004) Annu Rev Cell Dev Biol 20:781-810; Bejsovec (2005) Cell 120:11-4.

In some embodiments, the canonical Wnt signaling pathway is induced by contacting the stem cells or progenitor cells with a Wnt ligand, e.g., a Wnt agonist. Suitable Wnt agonists include an agonist of one or more of Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16. See, e.g., U.S. Patent Publication No. 2006/0147435. In some embodiments, Wnt ligand is soluble Wnt3a. In some embodiments, the Wnt agonist is a wnt polypeptide, a dishevelled polypeptide, or a β-catenin polypeptide. In some embodiments, the inducing or contacting step occurs before mesoderm commitment.

In some embodiments, a subject method involves contacting a stem cell or progenitor cell population with a Wnt3a polypeptide. Wnt3a polypeptides are known in the art, and any Wnt3a polypeptide that is capable of functioning as an inducer of a canonical Wnt signaling pathway can be used. In some embodiments, a Wnt3a polypeptide suitable for use in a subject method comprises an amino acid sequence having at least about 75%, at least about 80%, at least about 85%, at least about 90%, at least about 95%, at least about 98%, or 100% amino acid sequence identity to the amino acid sequence set forth in SEQ ID NO: 1. In some embodiments, a Wnt3a polypeptide suitable for use in a subject method comprises an amino acid sequence having at least about 75%, at least about 80%, at least about 85%, at least about 90%, at least about 95%, at least about 98%, or 100% amino acid sequence identity to a contiguous stretch of from about 250 amino acids to about 275 amino acids, from about 275 amino acids to about 300 amino acids, from about 300 amino acids to about 325 amino acids, or from about 325 amino acids to about 350 amino acids, of the amino acid sequence set forth in SEQ ID NO: 1. In some embodiments, a Wnt3a polypeptide suitable for use in a subject method comprises an amino acid sequence having at least about 75%, at least about 80%, at least about 85%, at least about 90%, at least about 95%, at least about 98%, or 100% amino acid sequence identity to amino acids 25-352 of the amino acid sequence set forth in SEQ ID NO:1, where the Wnt3a polypeptide lacks a signal sequence (e.g., a signal sequence corresponding to amino acids 1-24 of the amino acid sequence set forth in SEQ ID NO:1). In some embodiments, a Wnt3a polypeptide suitable for use in a subject method comprises an amino acid sequence having at least about 75%, at least about 80%, at least about 85%, at least about 90%, at least about 95%, at least about 98%, or 100% amino acid sequence identity to a contiguous stretch of from about 250 amino acids to about 275 amino acids, from about 275 amino acids to about 300 amino acids, or from about 300 amino acids to about 328 amino acids of the amino acid sequence set forth in amino acids 25-352 of SEQ ID NO:1.

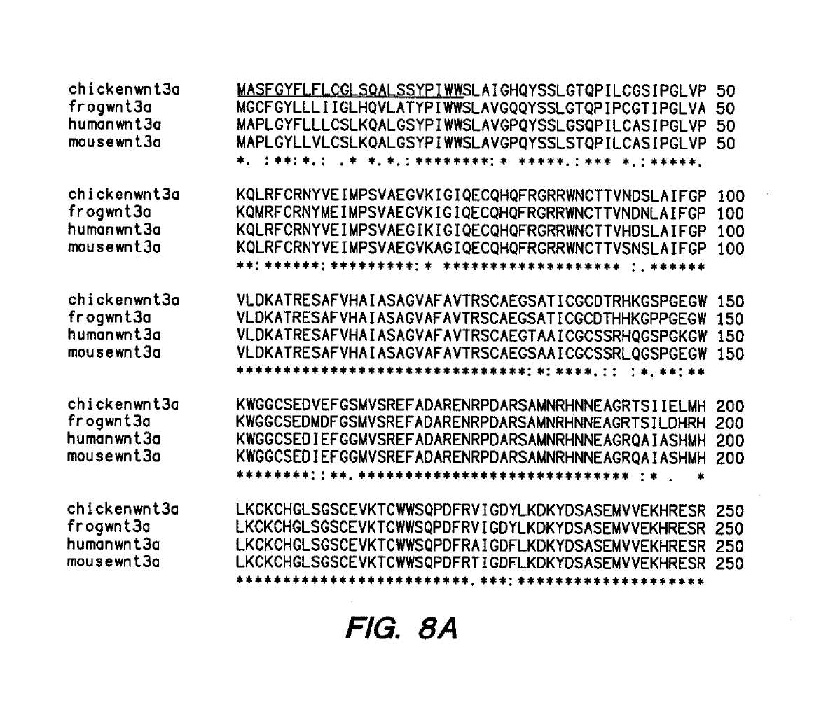

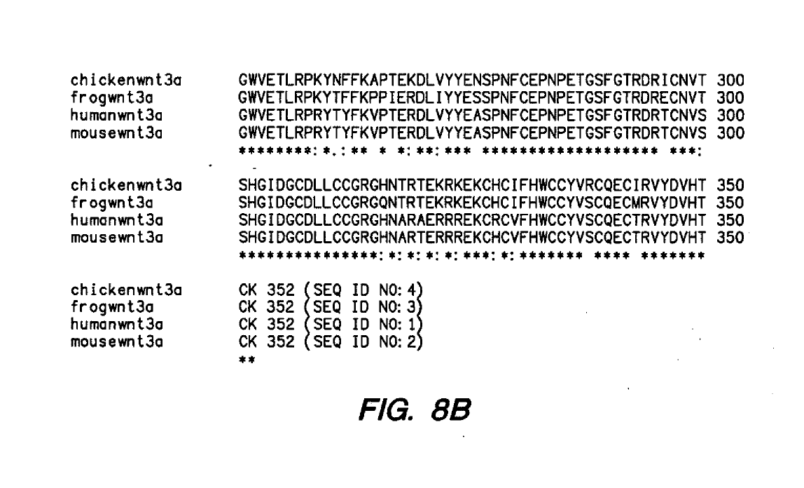

An alignment of amino acid sequences of human (GenBank Accession No. NP_149122; SEQ ID NO:1), mouse (GenBank Accession No. NP_033548; SEQ ID NO:2), chicken (GenBank Accession No. AAY87456; SEQ ID NO:4), and frog (GenBank Accession No. NP_001079343; SEQ ID NO:3) is shown in FIGS. 8A and 8B. The underlined sequence is a signal peptide. The 23 conserved cysteine residues are depicted in bold text. The alignment shows amino acids that are conserved among human, mouse, chicken, and frog Wnt3a. Amino acids that are can be varied in a given Wnt3a protein include, e.g., amino acid 132 (e.g., where amino acid 132 can be a threonine or a serine); amino acid 134 (e.g., where amino acid 134 can be an alanine or a threonine); amino acid 140 (e.g., where amino acid 140 can be a threonine or a serine); amino acid 143 (e.g., where amino acid 143 can be a lysine or a glutamine); amino acid 194 (e.g., where amino acid 194 can be a serine or an alanine); amino acid 230 (e.g., where amino acid 230 can be a tyrosine or a phenylalanine); amino acid 260 (e.g., where amino acid 260 can be a tyrosine or a phenylalanine); amino acid 272 (e.g., where amino acid 272 can be an isoleucine or a valine); amino acid 300 (e.g., where amino acid 300 can be a threonine or a serine); amino acid 320 (e.g., where amino acid 320 can be a threonine or an alanine); and amino acid 330 (e.g., where amino acid 330 can be an isoleucine or a valine).

In some embodiments, a suitable Wnt3a polypeptide is a fusion protein, where a Wnt3a fusion protein comprises a Wnt3a polypeptide fused to a heterologous protein (a “fusion partner). Suitable heterologous proteins include epitope tags; polypeptides that provide for solubility of the Wnt3a fusion protein; polypeptides that increase the stability of the Wnt3a fusion protein; polypeptides that provide detectable signals; and the like. Polypeptides that provide detectable signals include, e.g., enzymes that act on substrates to yield a directly detectable product such as a luminescent product, a colored product, a fluorescent product, etc.; fluorescent proteins, e.g., green fluorescent proteins, yellow fluorescent proteins, red fluorescent proteins, and the like; etc.

In some embodiments, a Wnt3a polypeptide is included in cell culture medium, in which the stem cells and/or progenitor cells are present, at a concentration of from about 5 ng per milliliter cell culture medium to about 5000 ng per milliliter culture medium. For example, in some embodiments, stem cells and/or progenitor cells are cultured in a medium comprising a Wnt3a polypeptide at a concentration of from about 5 ng/ml to about 10 ng/ml, from about 10 ng/ml to about 25 ng/ml, from about 25 ng/ml to about 50 ng/ml, from about 50 ng/ml to about 100 ng/ml, from about 100 ng/ml to about 250 ng/ml, from about 250 ng/ml to about 500 ng/ml, from about 500 ng/ml to about 750 ng/ml, from about 750 ng/ml to about 1000 ng/ml, from about 1000 ng/ml to about 2000 ng/ml, from about 2000 ng/ml to about 3000 ng/ml, from about 3000 ng/ml to about 4000 ng/ml, or from about 4000 ng/ml to about 5000 ng/ml, or more than 5000 ng/ml, cell culture medium.

### Increasing the Level of β-Catenin

The present invention provides methods of inducing cardiomyogenesis and/or expansion of cardiac progenitors in a population of stem cells or progenitor cells. In some embodiments, the method comprises increasing the level of β-catenin in the stem cells or progenitor cells.

In some embodiments, the method generally involves genetically modifying the stem cells or progenitor cells with an expression construct that comprises a nucleotide sequence encoding β-catenin, wherein the encoded β-catenin is produced in the stem cells or progenitor cells, where the β-catenin levels in the genetically modified stem cells or progenitor cells is higher than the level of β-catenin in stem cells or progenitor cells not genetically modified with the expression construct, and where the higher levels of β-catenin induce cardiomyogenesis. In some embodiments, the expression construct is a viral construct, e.g., a recombinant adeno-associated virus construct, a recombinant adenoviral construct, a recombinant lentiviral construct, etc.

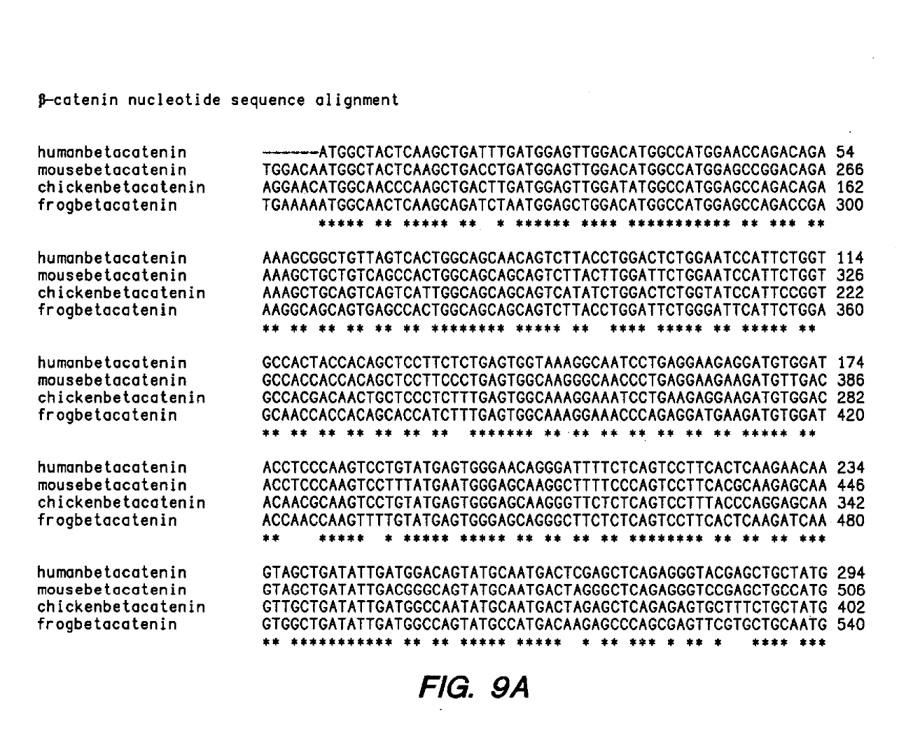

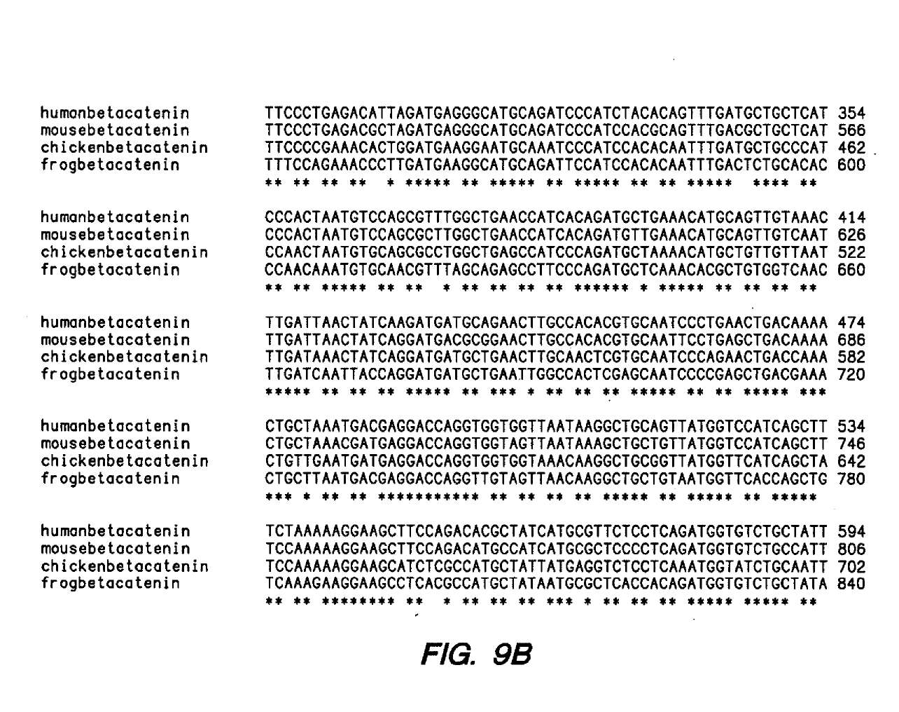

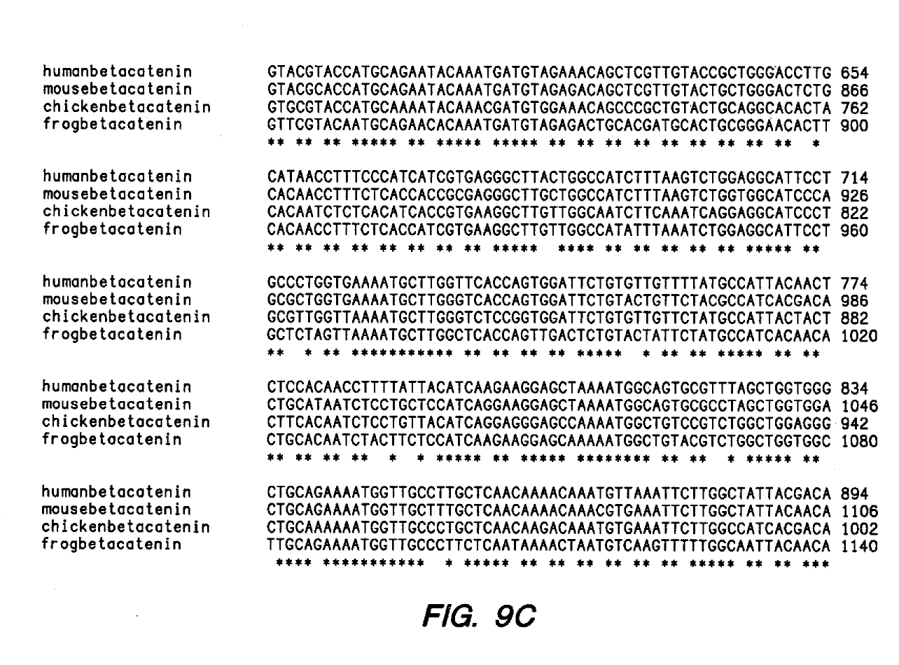

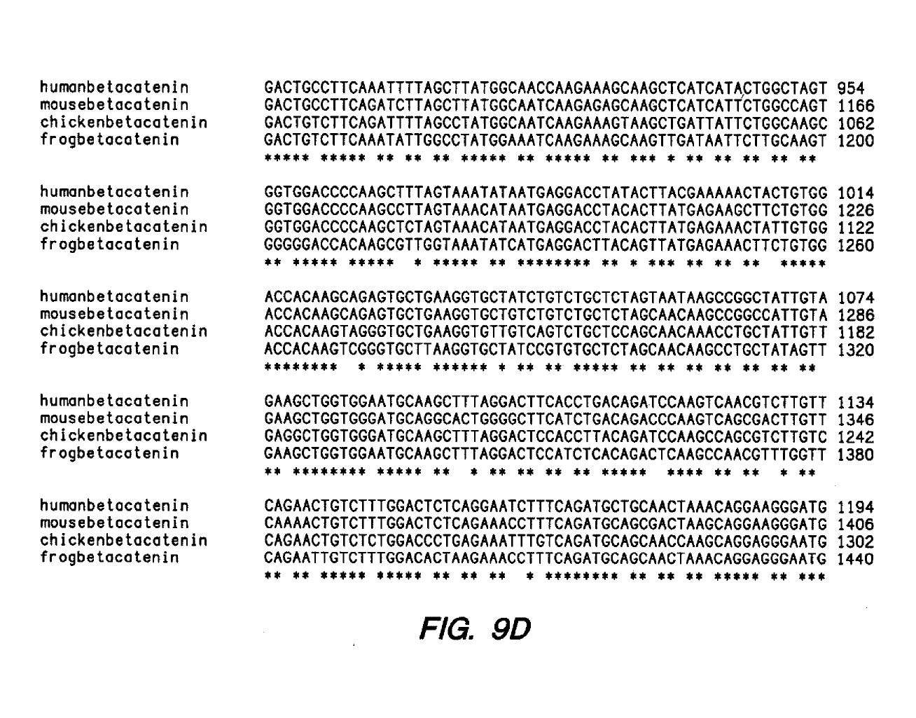

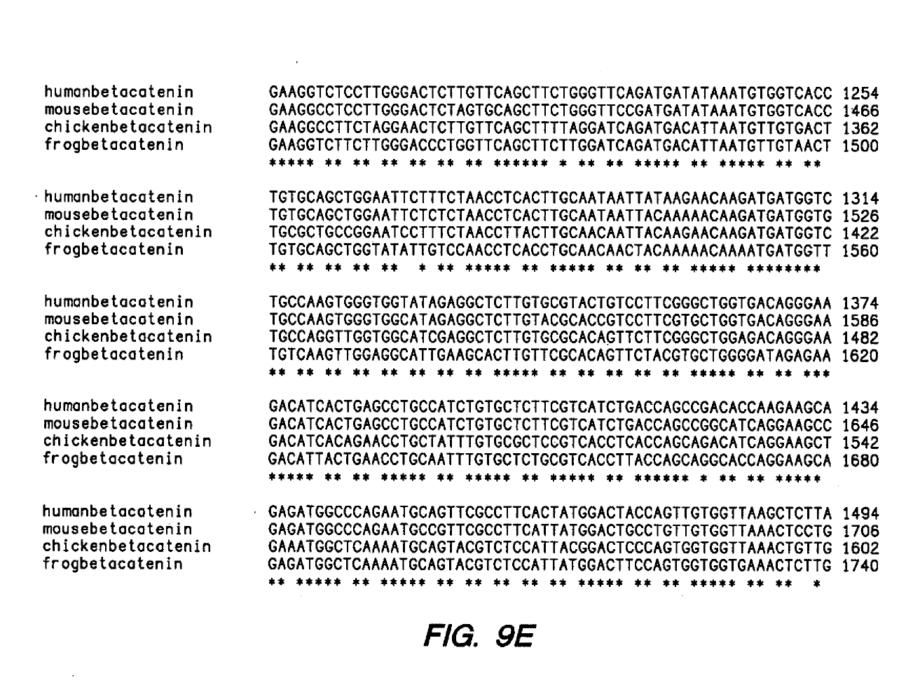

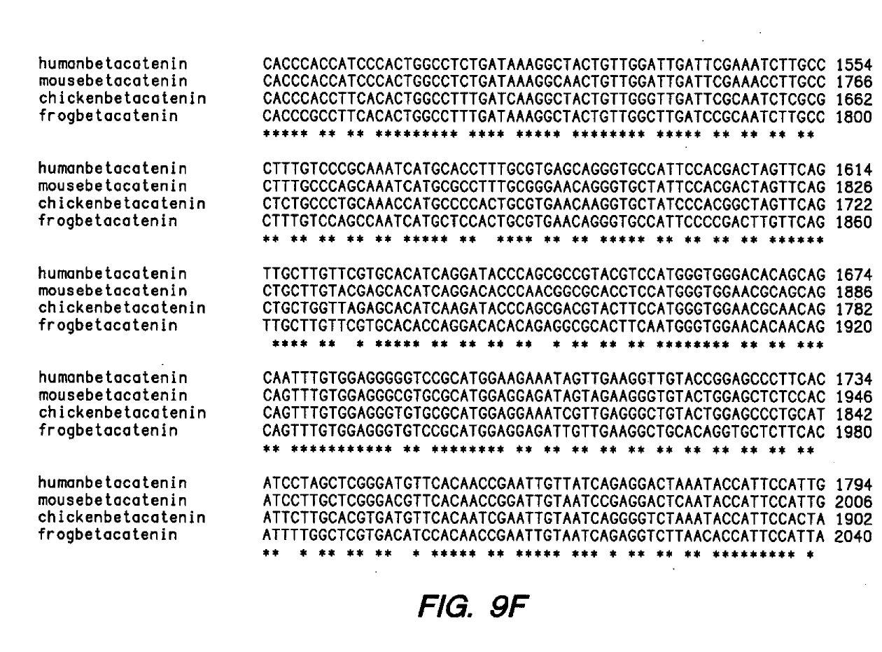

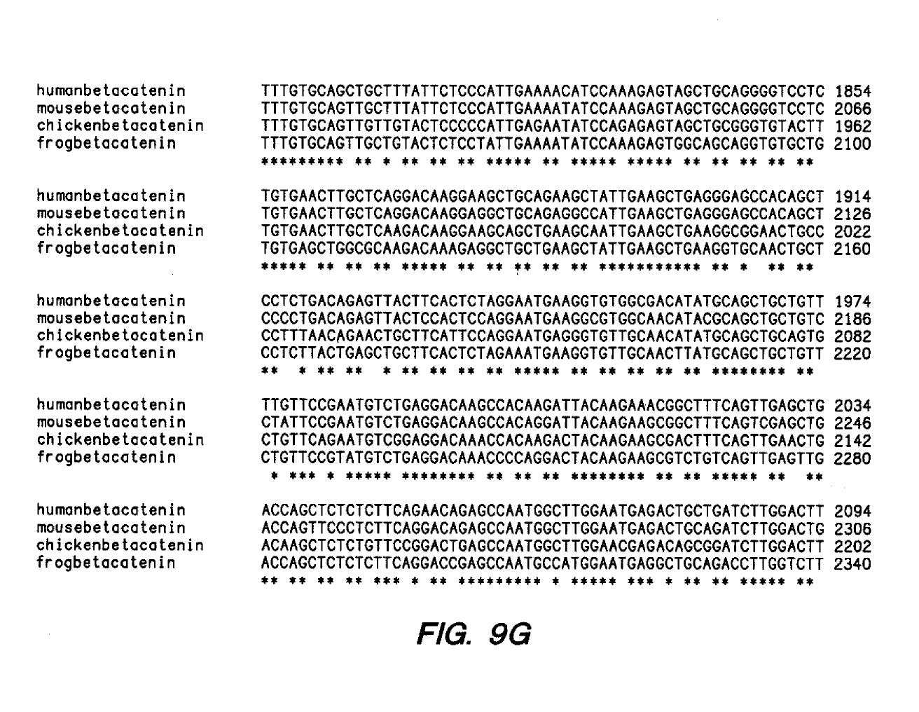

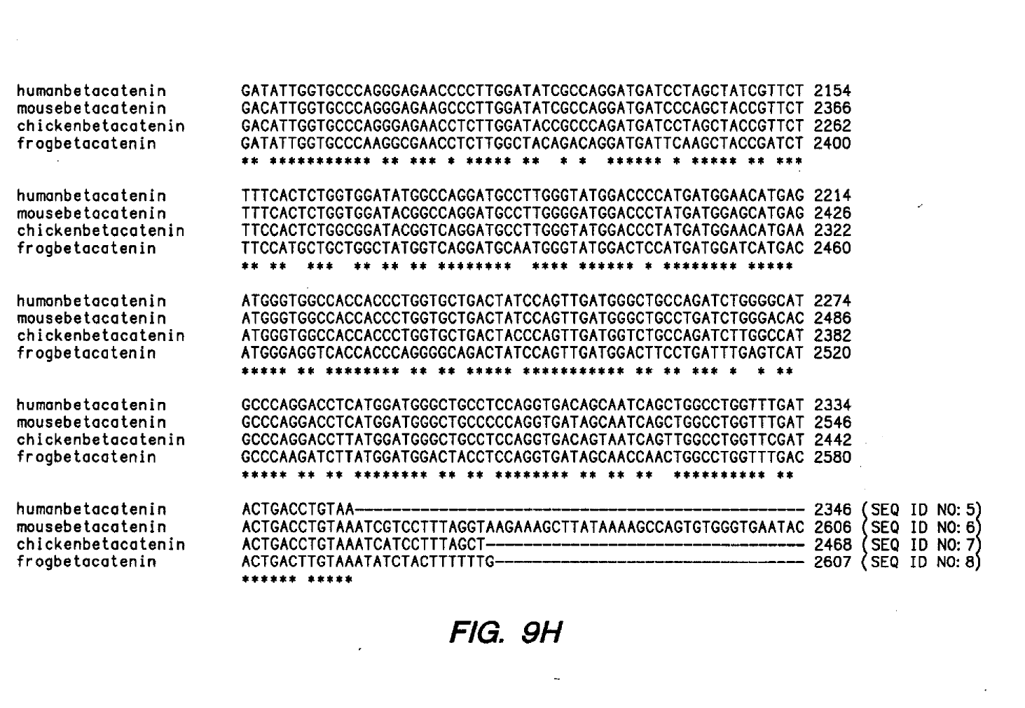

Nucleotide sequences encoding β-catenin are known in the art, and any nucleotide sequence that encodes a functional β-catenin is suitable for use. In some embodiments, a suitable polynucleotide comprises a nucleotide sequence having at least about 75%, at least about 80%, at least about 85%, at least about 90%, at least about 95%, at least about 98%, at least about 99%, or 100% nucleotide sequence identity to the nucleotide sequence set forth in SEQ ID NO:5 (GenBank Accession No. X87838; FIGS. 9A-H). In some embodiments, a suitable polynucleotide comprises a nucleotide sequence having at least about 75%, at least about 80%, at least about 85%, at least about 90%, at least about 95%, at least about 98%, at least about 99%, or 100% nucleotide sequence identity to a contiguous stretch of from about 1500 nucleotides to about 1750 nucleotides, from about 1750 nucleotides to about 2000 nucleotides, or from about 2000 nucleotides to about 2346 nucleotides of the nucleotide sequence set forth in SEQ ID NO:5.

FIGS. 9A-H provide a nucleotide sequence alignment of nucleotide sequences encoding human β-catenin (GenBank Accession No. X87838; SEQ ID NO:5), mouse β-catenin (GenBank Accession No. BC053065; SEQ ID NO:6), chicken β-catenin (GenBank Accession No. U82964; SEQ ID NO:7), and frog β-catenin (BC108764; SEQ ID NO:8). From the sequence alignment, changes that can be made to the nucleotide sequence are readily apparent.

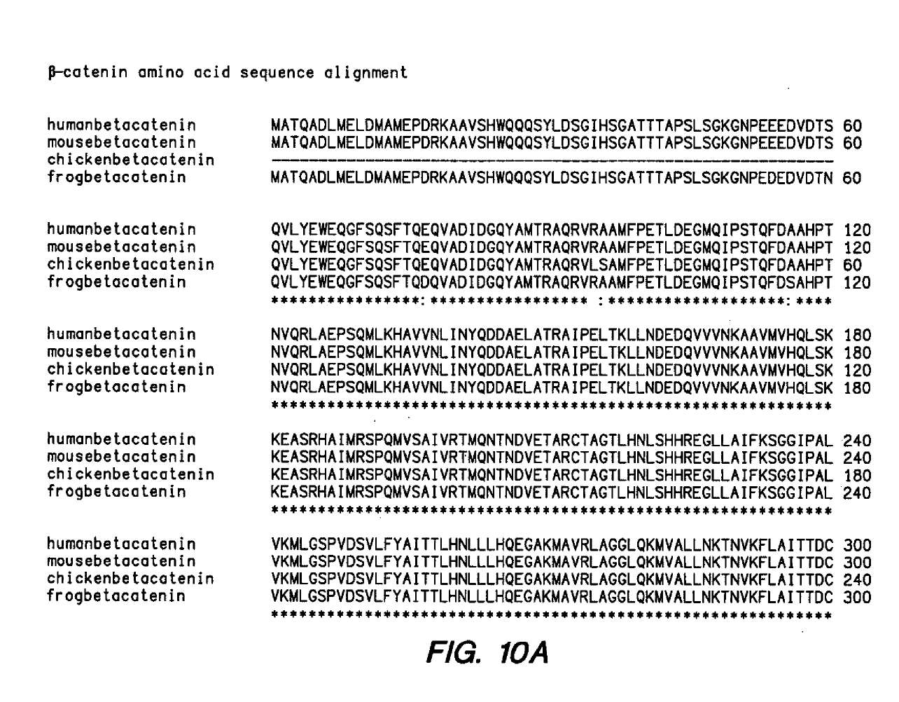

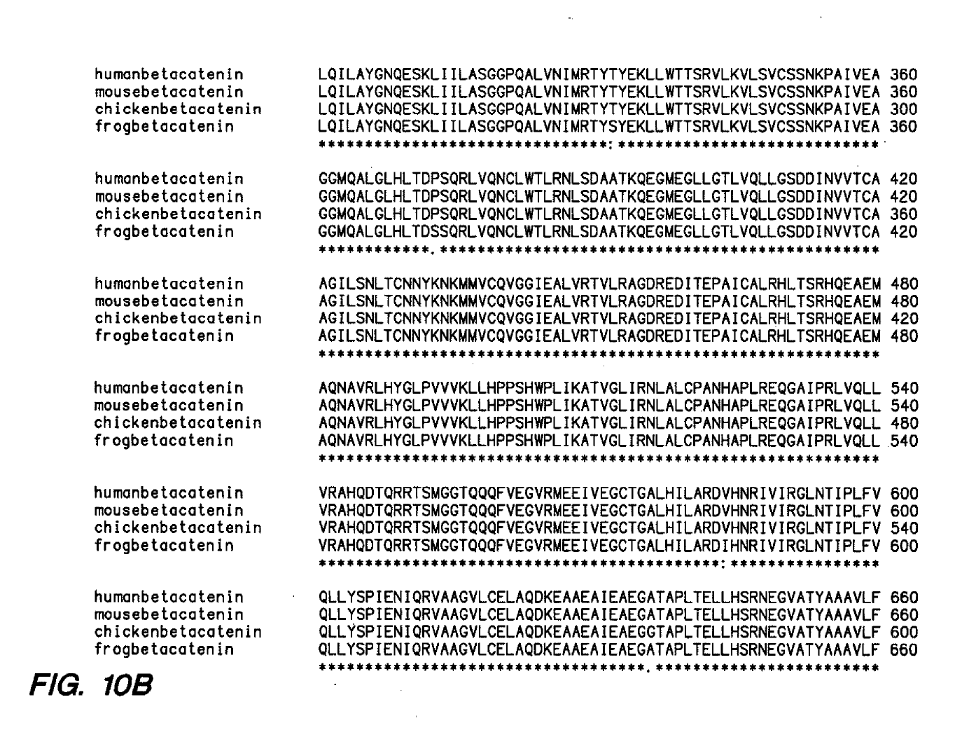

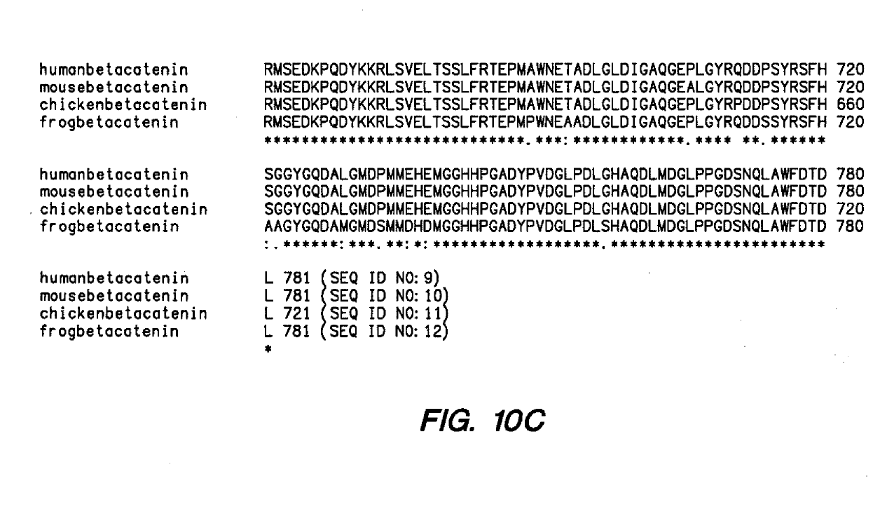

FIGS. 10A-C provide an amino acid sequence alignment of amino acid sequences of human β-catenin (GenBank Accession No. CAA61107; SEQ ID NO:9), mouse β-catenin (GenBank Accession No. AAH53065; SEQ ID NO:10), chicken β-catenin (GenBank Accession No. AAB80856; SEQ ID NO: 11), and frog β-catenin (GenBank Accession No. AA108765; SEQ ID NO:12).

In some embodiments, a suitable polynucleotide is one that comprises an amino acid sequence that encodes a polypeptide having at least about 75%, at least about 80%, at least about 85%, at least about 90%, at least about 95%, at least about 98%, at least about 99%, or 100% nucleotide sequence identity to the amino acid sequence set forth in SEQ ID NO:9. In some embodiments, a suitable polynucleotide is one that comprises an amino acid sequence that encodes a polypeptide having at least about 75%, at least about 80%, at least about 85%, at least about 90%, at least about 95%, at least about 98%, at least about 99%, or 100% nucleotide sequence identity to a contiguous stretch of from about 600 amino acids to about 650 amino acids, from about 650 amino acids to about 700 amino acids, from about 700 amino acids to about 750 amino acids, or from about 750 amino acids to about 781 amino acids, of the amino acid sequence set forth in SEQ ID NO:9.

In some embodiments, the expression construct is a viral construct, e.g., a recombinant adeno-associated virus construct (see, e.g., U.S. Pat. No. 7,078,387), a recombinant adenoviral construct, a recombinant lentiviral construct, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXTI, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

Non-limiting examples of suitable eukaryotic promoters (promoters functional in a eukaryotic cell) include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

In some embodiments, the β-catenin-encoding nucleotide sequence is operably linked to a cardiac-specific transcriptional regulator element (TRE), where TREs include promoters and enhancers. Suitable TREs include, but are not limited to, TREs derived from the following genes: myosin light chain-2, α-myosin heavy chain, AE3, cardiac troponin C, and cardiac actin. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Methods of introducing a nucleic acid into a host cell are known in the art, and any known method can be used to introduce a nucleic acid (e.g., an expression construct comprising a nucleotide sequence encoding a β-catenin polypeptide) into a stem cell or progenitor cell. Suitable methods include, e.g., infection, lipofection, electroporation, calcium phosphate precipitation, DEAE-dextran mediated transfection, liposome-mediated transfection, and the like.

Separating Cardiomyocytes from a Mixed Cell Population

In some embodiments, a subject method comprises: a) inducing cardiomyogenesis in a population of stem cells or progenitor cells, generating a mixed population of undifferentiated stem cells and/or undifferentiated progenitor cells and cardiomyocytes; and b) separating cardiomyocytes from the undifferentiated (non-cardiomyocyte) cells. In some embodiments, the separation step comprises contacting the cells with an antibody specific for a cardiomyocyte-specific cell surface marker. Suitable cardiomyocyte-specific cell surface markers include, but are not limited to, troponin and tropomyosin.

Separation can be carried out using well-known methods, including, e.g., any of a variety of sorting methods, e.g., fluorescence activated cell sorting (FACS), negative selection methods, etc. The selected cells are separated from non-selected cells, generating a population of selected (“sorted”) cells. A selected cell population can be at least about 75%, at least about 80%, at least about 85%, at least about 90%, at least about 95%, at least about 98%, at least about 99%, or greater than 99% cardiomyocytes.

Cell sorting (separation) methods are well known in the art. Procedures for separation may include magnetic separation, using antibody-coated magnetic beads, affinity chromatography and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. Dead cells may be eliminated by selection with dyes associated with dead cells (propidium iodide [PI], LDS). Any technique may be employed which is not unduly detrimental to the viability of the selected cells. Where the selection involves use of one or more antibodies, the antibodies can be conjugated with labels to allow for ease of separation of the particular cell type, e.g. magnetic beads; biotin, which binds with high affinity to avidin or streptavidin; fluorochromes, which can be used with a fluorescence activated cell sorter; haptens; and the like. Multi-color analyses may be employed with the FACS or in a combination of immunomagnetic separation and flow cytometry.

### Utility

A subject method is useful for generating a population of cardiomyocytes or cardiac progenitors, which cardiomyocytes or cardiac progenitors can be used in research applications, for generating artificial heart tissue, and in treatment methods.

### Research Applications

A subject method can be used to generate cardiomyocytes or cardiac progenitors for research applications. Research applications include, e.g., introduction of the cardiomyocytes or cardiac progenitors into a non-human animal model of a disease (e.g., a cardiac disease) to determine efficacy of the cardiomyocytes or cardiac progenitors in the treatment of the disease; use of the cardiomyocytes in screening methods to identify candidate agents suitable for use in treating cardiac disorders; and the like. For example, a cardiomyocyte or cardiac progenitor generated using a subject method can be contacted with a test agent, and the effect, if any, of the test agent on a biological activity of the cardiomyocyte or cardiac progenitor can be assessed, where a test agent that has an effect on a biological activity of the cardiomyocyte or cardiac progenitor is a candidate agent for treating a cardiac disorder. As another example, a cardiomyocyte or cardiac progenitor generated using a subject method can be introduced into a non-human animal model of a cardiac disorder, and the effect of the cardiomyocyte or cardiac progenitor on ameliorating the disorder can be tested in the non-human animal model.

### Treatment Methods

A subject method is useful for generating artificial heart tissue, e.g., for implanting into a mammalian subject in need thereof. A subject method is useful for replacing damaged heart tissue (e.g., ischemic heart tissue). A subject method is useful for stimulating endogenous stem cells resident in the heart to undergo cardiomyogenesis. Where a subject method involves introducing (implanting) a cardiomyocyte into an individual, allogenic or autologous transplantation can be carried out.

The present invention provides methods of treating a cardiac disorder in an individual, the method generally involving administering to an individual in need thereof a therapeutically effective amount of one or more of: a) an agent that induces canonical Wnt signaling; b) an expression construct that comprises a nucleotide sequence encoding β-catenin; c) a population of cardiomyocytes prepared using a subject method; d) a population of cardiac progenitors prepared using a subject method; and e) an artificial heart tissue prepared using a subject method. Individuals in need of treatment using a subject method include, but are not limited to, individuals having a congenital heart defect; individuals suffering from a condition that results in ischemic heart tissue, e.g., individuals with coronary artery disease; and the like. A subject method is useful to treat degenerative muscle disease, e.g., familial cardiomyopathy, dilated cardiomyopathy, hypertrophic cardiomyopathy, restrictive cardiomyopathy, or coronary artery disease with resultant ischemic cardiomyopathy.

Individuals who are suitable for treatment with a subject method include individuals (e.g., mammalian subjects, such as humans; non-human primates; experimental non-human mammalian subjects such as mice, rats, etc.) having a cardiac condition, e.g., a condition that results in ischemic heart tissue, e.g., individuals with coronary artery disease; and the like. Individuals suitable for treatment with a subject method include individuals who have a degenerative muscle disease, e.g., familial cardiomyopathy, dilated cardiomyopathy, hypertrophic cardiomyopathy, restrictive cardiomyopathy, or coronary artery disease with resultant ischemic cardiomyopathy.

For administration to a mammalian host, a cardiomyocyte population or cardiac progenitor cell population generated using a subject method can be formulated as a pharmaceutical composition. A pharmaceutical composition can be a sterile aqueous or non-aqueous solution, suspension or emulsion, which additionally comprises a physiologically acceptable carrier (i.e., a non-toxic material that does not interfere with the activity of the cardiomyocytes). Any suitable carrier known to those of ordinary skill in the art may be employed in a subject pharmaceutical composition. The selection of a carrier will depend, in part, on the nature of the substance (i.e., cells or chemical compounds) being administered. Representative carriers include physiological saline solutions, gelatin, water, alcohols, natural or synthetic oils, saccharide solutions, glycols, injectable organic esters such as ethyl oleate or a combination of such materials. Optionally, a pharmaceutical composition may additionally contain preservatives and/or other additives such as, for example, antimicrobial agents, anti-oxidants, chelating agents and/or inert gases, and/or other active ingredients.

In some embodiments, a cardiomyocyte population or cardiac progenitor population is encapsulated, according to known encapsulation technologies, including microencapsulation (see, e.g., U.S. Pat. Nos. 4,352,883; 4,353,888; and 5,084,350). Where the cardiomyocytes or cardiac progenitors are encapsulated, in some embodiments the cardiomyocytes or cardiac progenitors are encapsulated by macroencapsulation, as described in U.S. Pat. Nos. 5,284,761; 5,158,881; 4,976,859; 4,968,733; 5,800,828 and published PCT patent application WO 95/05452.

In some embodiments, a cardiomyocyte population or cardiac progenitor population is present in a matrix, as described below.

A unit dosage form of a cardiomyocyte population or cardiac progenitor population can contain from about 10³ cells to about 10⁹ cells, e.g., from about 10³ cells to about 10⁴ cells, from about 10⁴ cells to about 10⁵ cells, from about 10⁵ cells to about 10⁶ cells, from about 10⁶ cells to about 10⁷ cells, from about 10⁷ cells to about 10⁸ cells, or from about 10⁸ cells to about 10⁹ cells.

A cardiomyocyte population can be cryopreserved according to routine procedures. For example, cryopreservation can be carried out on from about one to ten million cells in “freeze” medium which can include a suitable proliferation medium, 10% BSA and 7.5% dimethylsulfoxide. Cells are centrifuged. Growth medium is aspirated and replaced with freeze medium. Cells are resuspended as spheres. Cells are slowly frozen, by, e.g., placing in a container at −80° C. Cells are thawed by swirling in a 37° C. bath, resuspended in fresh proliferation medium, and grown as described above.

### Artificial Heart Tissue

In some embodiments, a subject method comprises: a) inducing cardiomyogenesis in a population of stem cells or progenitor cells in vitro, e.g., where the stem cells or progenitor cells are present in a matrix, wherein a population of cardiomyocytes is generated; and b) implanting the population of cardiomyocytes into or on an existing heart tissue in an individual. Thus, the present invention provides a method for generating artificial heart tissue in vitro; and implanting the artificial heart tissue in vivo.

The artificial heart tissue can be used for allogenic or autologous transplantation into an individual in need thereof. To produce artificial heart tissue, a matrix can be provided which is brought into contact with the stem cells or progenitor cells, where the stem cells or progenitor cells are induced to undergo cardiomyogenesis using a subject method, as described above. This means that this matrix is transferred into a suitable vessel and a layer of the cell-containing culture medium is placed on top (before or during the differentiation of the expanded stem cells or progenitor cells). The term “matrix” should be understood in this connection to mean any suitable carrier material to which the cells are able to attach themselves or adhere in order to form the corresponding cell composite, i.e. the artificial tissue. In some embodiments, the matrix or carrier material, respectively, is present already in a three-dimensional form desired for later application. For example, bovine pericardial tissue is used as matrix which is crosslinked with collagen, decellularized and photofixed.

For example, a matrix (also referred to as a “biocompatible substrate”) is a material that is suitable for implantation into a subject onto which a cell population can be deposited. A biocompatible substrate does not cause toxic or injurious effects once implanted in the subject. In one embodiment, the biocompatible substrate is a polymer with a surface that can be shaped into the desired structure that requires repairing or replacing. The polymer can also be shaped into a part of a structure that requires repairing or replacing. The biocompatible substrate provides the supportive framework that allows cells to attach to it, and grow on it. Cultured populations of cells can then be grown on the biocompatible substrate, which provides the appropriate interstitial distances required for cell-cell interaction.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Inducing Cardiomyogenesis in Embryonic Stem Cells

Guiding stem or progenitor cells into distinct lineages remains a fundamental challenge in stem cell biology. Members of the Wnt pathway control many pivotal embryonic events (Jessen and Solnica-Krezel Cell 120, 736-7 (2005); Olson Cell 125, 593-605 (2006); Karner et al. Semin Cell Dev Biol 17, 214-22 (2006)), and canonical Wnt ligands are thought to inhibit mesodermal progenitors from differentiating into cardiomyocytes in some species (Schneider and Mercola Genes Dev 15, 304-15 (2001); Marvin et al. Genes Dev 15, 316-27 (2001); Tzahor and Lassar Genes Dev 15, 255-60 (2001)). However, the cell-autonomous role of canonical Wnt signaling through its obligatory transcriptional mediator, β-catenin, is unknown. Here, it is shown that early cardiac progenitors are enriched in β-catenin protein in vivo and that mice lacking β-catenin in cardiac progenitor cells have fewer cardiomyocytes and ventricular hypoplasia. Cardiomyocytes lacking β-catenin had a proliferative defect, and the Wnt/β-catenin target gene cyclin D2 was downregulated in mutant hearts. Conversely, stabilization of β-catenin in the heart resulted in excessive proliferation of myocytes and upregulation of cyclin D2. At discrete windows of development in embryonic stem cells, activation of canonical Wnt signaling promoted cardiomyogenesis, and inhibition of canonical Wnts repressed cardiac differentiation. These findings demonstrate that canonical Wnt signaling promotes some of the earliest events of cardiogenesis and positively regulates cardiomyocyte differentiation in embryonic stem cells.

### Materials and Methods

Mouse Lines and Genetics.

The Nkx2.5-cre, ctnnb1^{tm2Kem} or Islet1-cre, ctnnb1^{tm2Kem} homozygous embryos were obtained by crossing Nkx2.5-cre, ctnnb1^{tm2Kem flox/+} or Islet1-cre, ctnnb1^{tm2Kem flox/+} lines with ctnnb1^{tm2Kem flox/flox} lines, respectively. Nkx2.5-cre, β-catenin/loxP(ex3) heterozygous embryos were obtained by crossing Nkx2.5-cre lines with β-catenin/loxP(ex3)^{flox/+} lines. Wild type and mutant embryos were identified as described. Jaspard et al. Mech Dev 90, 263-7 (2000); and Brault et al. Development 128, 1253-64 (2001).

Immunohistochemistry and In Situ Hybridization.

Whole or cryosectioned embryos and EBs were stained with the following antibodies: mouse anti-Islet1 (1:200) and anti-tropomyosin (1:100) (DSHB), goat anti-Wnt8a (15 μg/ml, R&D Systems), goat anti-β-catenin (1:50) and rat anti-cyclin D2 (1:100) (Santa Cruz Biotechnology), rabbit anti-phospho-Histone H3 (1:100, Upstate), and horseradish peroxidase-conjugated (with TSA plus Fluorescent Systems, PerkinElmer) secondary antibodies (The Jackson Laboratory). mRNA in situ hybridization was performed with designated antisense probes as described. Srivastava et al Science 270, 1995-9 (1995).

ES Cell Culture and Gene Expression Analysis.

Murine ES cells were propagated undifferentiated in maintenance medium consisting of Glasgow MEM (Sigma G5154) supplemented with 10% fetal bovine serum (FBS, Hyclone SH30071.03), 1 mM β-mercaptoethanol (Sigma M7522), 2 mM L-glutamine (Gibco-BRL 25030-081), 1 mM sodium pyruvate (Gibco-BRL 11360-070), 0.1 mM minimum essential medium containing nonessential amino acids, and LIF-conditioned medium (1:1000). EBs were formed by culturing ES cells (6×10⁵/well) for 3 days in ultra-low-attachment six-well plates (Costar 07200601) in differentiation medium (DM), which contained the same components as maintenance medium but had 20% FBS and no LIF. For early treatment, the medium was replaced with DM containing Wnt3a (150 ng/ml, R&D systems) or Dkk1 (500 ng/ml, R&D systems) at the start of day 4; on day 6, the medium was replaced with fresh DM. For late treatment, DM was replaced with Wnt3a- or Dkk1-containing DM at the start of day 7 and switched to fresh DM on day 9. Respective media were changed for early-, late-, and untreated EBs every 3 days. EBs were monitored for beating from days 8-12. EBs were collected between days 3 and 12 at regular intervals. For gene expression analysis, total RNA was isolated, and real-time PCR was performed with the ABI Prism system (7900HT, Applied Biosystems). TaqMan primers used in this study are listed in Table 1. All samples were run in triplicate. Real-time PCR data were normalized and standardized with SDS2.2 software.

TABLE 1

| Gene Name | Gene ID | Species | ABI Order # |
|---|---|---|---|
| Brachyury (T) | NM_009309.1 | mus | Mm00436877_ml |
| Gapdh | NM_001001303 | mus | Mm99999915_gl |
| Gata4 | NM_008092 | mus | Mm00484689_ml |
| Hand1 | NM_008213.1 | mus | Mm00433931_ml |
| Hand2 | NM_010402.2 | mus | Mm00439247_ml |
| Islet1 | NM_021459.2 | mus | Mm00627860_ml |
| Mespl | NM_008588.1 | mus | Mm00801883_gl |
| Myh6 | NM_010856.2 | mus | Mm00440354_ml |
| Myh7 | NM_080728 | mus | Mm00600555_ml |
| Mlc2a | NM_022879.1 | mus | Mm00491655_ml |
| Mlc2v | NM_010861.2 | mus | Mm00440384_ml |
| Nkx2.5 | NM_008700.2 | mus | Mm00657783_ml |
| Tbx5 | NM_011537.2 | mus | Mm00803521_ml |

### Results

Soon after gastrulation commences, vertebrate heart formation begins in the anterior mesoderm, stimulated by the interplay of inductive and repressive signals. Olson and Schneider Genes Dev 17, 1937-56 (2003); Zaffran and Frasch Circ Res 91, 457-69 (2002). In response to signals from the neighboring endoderm and ectoderm, two domains of mesoderm progenitors—the first and second heart fields—adopt a cardiac fate, each contributing to distinct cardiac regions. Buckingham et al. Nat Rev Genet 6, 826-35 (2005); Srivastava Cell 126, 1037-48 (2006). Despite the conservation of cardiac developmental pathways across species, conflicting roles of Wnt signaling in cardiogenesis have been reported.

In flies, Wingless, the founding member of the canonical Wnt family, is required for cardiac lineage determination (Wu et al. Dev Biol 169, 619-28 (1995); Jagla et al. Development 124, 91-100 (1997)); however, in frogs and chick, overexpression of canonical Wnts inhibits cardiomyocyte commitment or differentiation, likely through effects on the endoderm. (Schneider and Mercola Genes Dev 15, 304-15 (2001); Marvin et al. Genes Dev 15, 316-27 (2001); Tzahor and Lassar Genes Dev 15, 255-60 (2001)). The canonical Wnt signaling pathway is initiated when Wnt ligands bind to Frizzled transmembrane receptors, thereby stabilizing protein levels of β-catenin, a transcriptional co-activator that interacts with the TCF/LEF family of transcription factors to activate Wnt target genes. Logan and Nusse Annu Rev Cell Dev Biol 20, 781-810 (2004); Bejsovec Cell 120, 11-4 (2005). Noncanonical Wnt signaling mediated by G protein-coupled receptor-dependent alterations in intracellular calcium may promote cardiogenesis (Pandur et al. Nature 418, 636-41 (2002)), although the cell-autonomous role of either Wnt pathway in mesodermal progenitors is unknown.

To determine if canonical Wnt signaling is active in the heart-forming region, β-catenin protein expression was examined in mouse embryos. At embryonic day (E) 8.5, the myocardial layer was enriched in β-catenin, suggesting active canonical Wnt signaling in cardiac progenitors. Of the canonical Wnt molecules expressed in developing heart (Wnt2, Wnt7a, Wnt8a) (Monkley et al. Development 122, 3343-53 (1996); Bond et al. Dev Dyn 227, 536-43 (2003); Jaspard et al. Mech Dev 90, 263-7 (2000)), it was found that Wnt8a was most specifically expressed in heart precursors at E8.5. β-catenin and Wnt8a expression persisted at later stages of development.

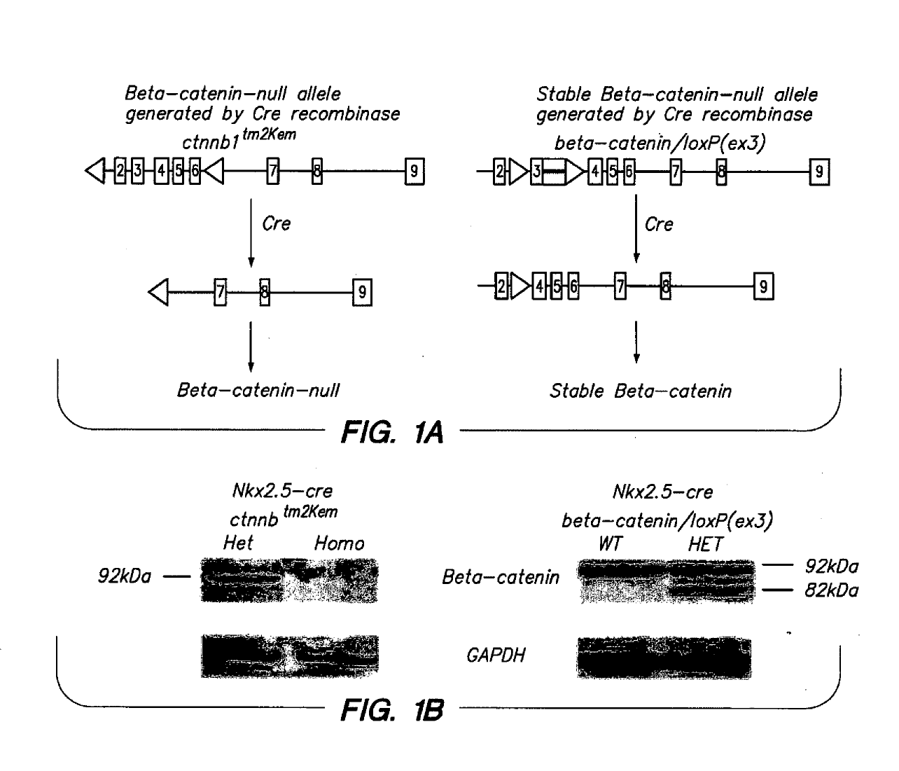

To examine the cell-autonomous role of canonical Wnt signaling in cardiomyocytes, β-catenin expression was disrupted or stabilized in cardiac mesodermal progenitors using ctnnb1^{tm2Kem} (Brault et al. Development 128, 1253-64 (2001)) or β-catenin/loxP(ex3) (Harada et al. EMBO J 18, 5931-42 (1999)) mice, respectively. In ctnnb1^{tm2Kem} mice, Cre recombinase-mediated excision of exons 2-6 produces a β-catenin-null allele; in β-catenin/loxP(ex3) mice, cre expression activates Wnt/β-catenin signaling by generating stable β-catenin (FIGS. 1A and 1B). The Nkx2.5-cre line was used to drive cre expression in ventricular cardiogenic progenitors beginning at E8.0, just after early cardiac commitment events have begun. McFadden et al. Development 132, 189-201 (2005). The Cre-mediated excision event was confirmed by Western analysis and was nearly complete (FIG. 1B).

Nkx2.5-cre, ctnnb1^{tm2Kem} homozygous embryos died around E12.5 from cardiac dysfunction, with significant reductions in ventricular size, particularly the right ventricle (RV), and fewer cardiomyocytes in the ventricular wall. In contrast, Nkx2.5-cre, β-catenin/loxP(ex3) heterozygous embryos with stabilized β-catenin had enlarged hearts with abnormally thickened ventricular walls, suggesting that canonical Wnt signaling regulates cardiomyocyte number.

To identify the source of the myocardial alterations, hearts were analyzed by immunostaining with anti-phospho-histone H3 (PH3) antibody to detect alterations in cell-cycle control or by TUNEL assay to detect apoptosis. PH3 staining was reduced (38%, P<0.01) in Nkx2.5-cre, ctnnb1^{tm2Kem} hearts and increased (132%, P<0.01) in Nkx2.5-cre, β-catenin/loxP(ex3) hearts. Levels of cyclin D2, a known target of Wnt/β-catenin signaling that promotes cell cycling (Kioussi et al. Cell 111, 673-85 (2002); Huang et al. Oncogene (2007) 26:2471), were increased in the Nkx2.5-cre, β-catenin/loxP(ex3) hearts and decreased in Nkx2.5-cre, ctnnb1^{tm2Kem} hearts. Apoptosis was unaffected. Thus, Wnt/β-catenin is required in derivatives of the first and second heart fields, which contribute to the left or right ventricles, respectively, to maintain cardiac progenitors in a proliferative state in a cell-autonomous fashion.

Since Nkx2.5-cre is not activated in undifferentiated mesodermal precursors, the preceding results likely represent a relatively late role for Wnt/β-catenin signaling after mesodermal progenitors become committed to the cardiac lineage. To assess its earlier role, β-catenin was deleted in second heart field progenitors before cardiac differentiation by crossing ctnnb1^{tm2Kem} mice with Islet1-cre mice, in which cre is expressed at E7.0-7.5 in mesodermal progenitors that give rise to the RV and outflow tract. Cai et al. Dev Cell 5, 877-89 (2003). In Islet1-cre, ctnnb1^{tm2Kem} homozygous embryos, the second heart field failed to form the RV, but the left ventricle (LV) was relatively normal in size. In situ hybridization with a probe recognizing the LV-specific transcript, Hand1 (Srivastava Trends Cardiovasc Med 9, 11-8 (1999)), also expressed in the outflow tract, confirmed the identity of the ventricular chamber. Sections of the mutant heart revealed a thin myocardial layer within the diminished RV and outflow tract domains, indicating a more severe defect of RV precursors than the later deletion with Nkx2.5-Cre. Consistent with the activity of Islet1-Cre in pharyngeal arch mesoderm, deletion of β-catenin led to hypoplasia of these structures as well. Thus, second heart field mesoderm progenitors may require Wnt/β-catenin signaling for specification or expansion. Islet1 expression was normal, suggesting that mesodermal progenitors were present but failed to differentiate into myocytes that contribute to the RV.

Although Wnt/β-catenin was required for cardiac progenitor cell development, our in vivo data do not establish whether Wnt/β-catenin signaling is required for induction of early cardiac progenitors nor can they address the initial molecular events regulated by Wnts. To answer these questions, the embryonic stem (ES) cell differentiation system, which allows access to progenitors at defined stages of development, was used. ES cells grown in hanging drops form embryoid bodies (EBs) that randomly differentiate into derivatives of the three germ layers and can form beating cardiomyocytes, although with low frequency. The frequency is even lower when EBs are grown in suspension, which is technically much easier and would allow higher throughput generation of cardiomyocytes from ES cells.

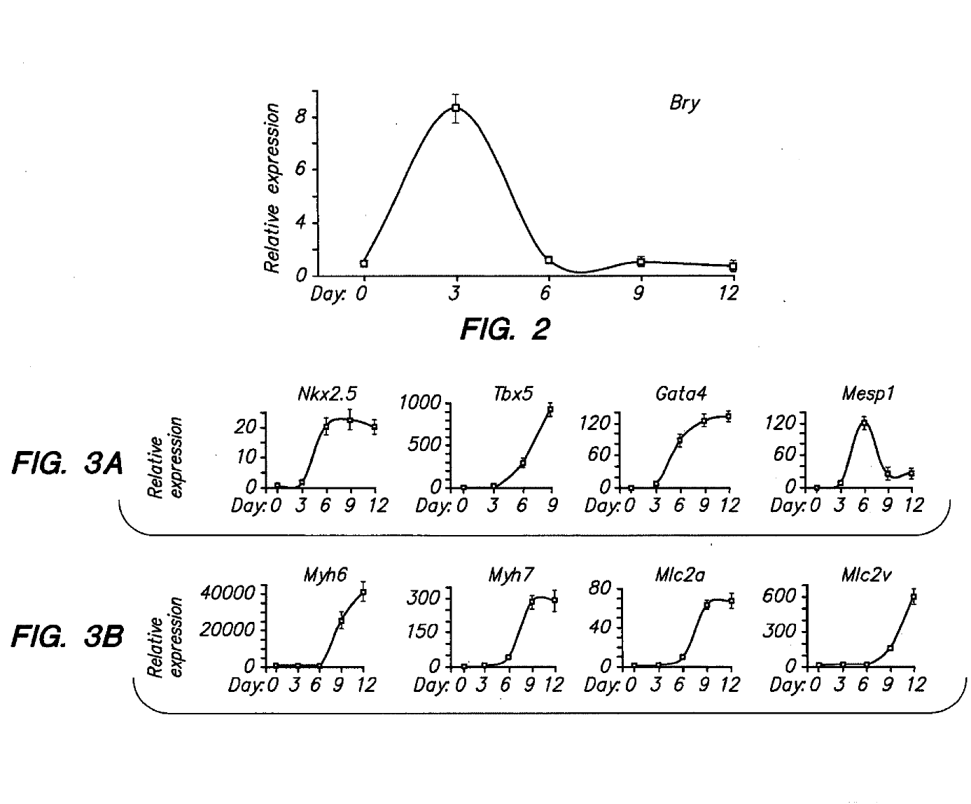

To determine the best time to target canonical Wnt signaling, mesoderm and cardiac gene expression was profiled during ES differentiation. The early mesoderm marker Brachyury (Bry) was strongly induced on day 3 of EB differentiation (EB3) but was downregulated afterward, suggesting mesoderm commitment by EB3 (FIG. 2). Other early cardiac mesoderm markers, including the transcription factors Nkx2.5, Tbx5, Gata4, and Mesp1, were highly induced between EB4-6. Late cardiac markers, including the sarcomeric genes Myh6, Myh7, Mlc2a, and Mlc2v, were induced by EB7-9 (FIGS. 3A and 3B).

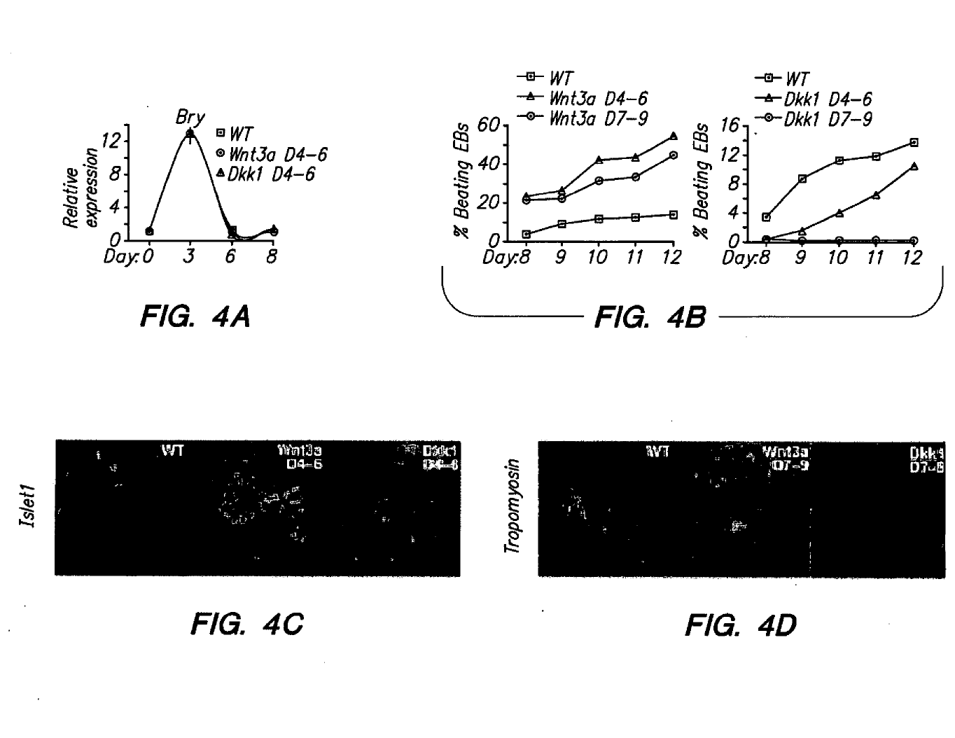

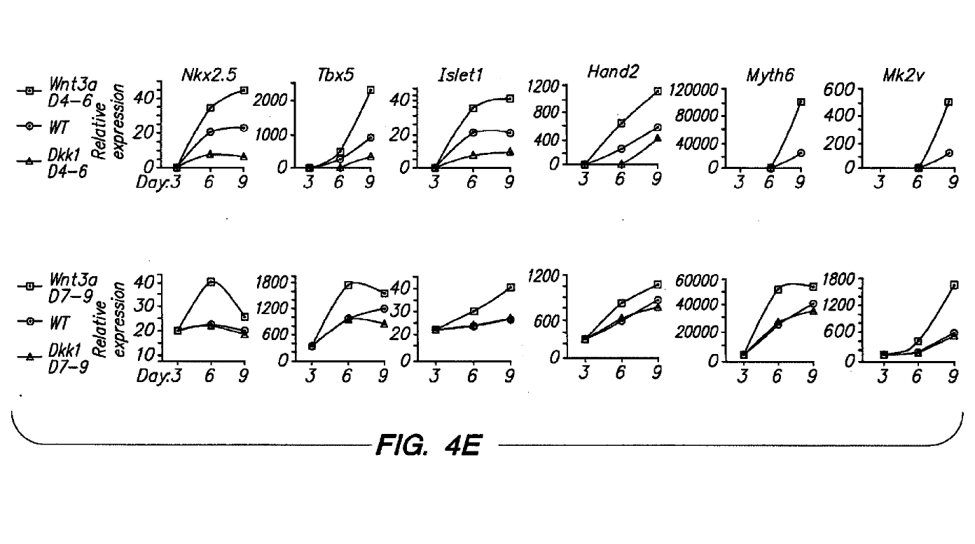

Canonical Wnt signaling is required for general mesoderm formation in vivo (Haegel et al. Development 121, 3529-37 (1995); Liu et al. Nat Genet 22, 361-5 (1999)) and in EBs (Lindsley et al. Development 133, 3787-96 (2006)). To facilitate interpretation of the effects of Wnt signaling specifically on cardiac differentiation, canonical Wnt signaling was targeted after mesoderm commitment (EB3) but before induction of early cardiac genes (EB4-5). Soluble Wnt3a, the soluble canonical Wnt ligand available, and dickopffl (Dkk1), an inhibitor of canonical Wnts, were used to promote or disrupt Wnt/β-catenin signaling. Bry expression was unaltered (FIG. 4A), suggesting that stimulating or inhibiting canonical Wnt signaling after EB3 did not alter the number of mesodermal progenitors. In contrast, stimulation of Wnt/I-catenin between EB4-6 with soluble Wnt3a increased the number of beating EBs at EB12 from 10% to over 50% when cells were grown in suspension (FIG. 4B, left, P<0.01). Inhibiting canonical Wnt signaling with Dkk-1 between EB4-6 resulted in a complete absence of beating EBs (FIG. 4B, right), suggesting that canonical Wnt signaling is required for cardiomyocyte formation in the ES cell differentiation system. Cardiac progenitors were similarly affected by Wnt signaling: the number of Isl1- and Tropomyosin-positive cells in EBs was increased by the Wnt agonist and decreased by the Wnt antagonist (FIGS. 4C and 4D).

Consistent with a positive role for Wnt in pushing mesodermal progenitors into the cardiac lineage, expression of the early cardiac genes Nkx2.5 and Tbx5 was upregulated by exposure to Wnt3a from EB4-6 and downregulated by similar exposure to Dkk-1 (FIG. 4E, top). The second heart field marker, Islet1, and the RV marker, Hand2 (Srivastava Trends Cardiovasc Med 9, 11-8 (1999)), showed similar responses to Wnt3a and Dkk-1, as did cardiac sarcomeric genes (FIG. 4E, top).

To assess effects of canonical Wnt signaling after induction of early cardiac mesoderm, EBs were treated with Wnt3a or Dkk-1 between EB 7-9. Nearly 50% of EBs treated with Wnt3a and grown in suspension were beating by EB 12 (FIG. 4B, left, P<0.01), and most early and late cardiac markers were upregulated (FIG. 4E, bottom), suggesting that Wnt had a positive effect on cardiac mesoderm differentiation even at this relatively late stage. Approximately 10% of EBs treated with Dkk-1 were beating (vs. 13% of control EBs, FIG. 4B, right, P>0.2), and cardiac gene expression was not changed (FIG. 4E, bottom). Thus, after cardiac mesoderm commitment, canonical Wnt signals appear to be dispensable for full cardiac differentiation but may still recruit mesodermal precursors or expand cardiac progenitors.

Misexpression or overexpression of canonical Wnt signaling agonists in model systems led to the prevailing view that inhibition of canonical Wnt signaling is required for vertebrate cardiogenesis (Olson and Schneider Genes Dev 17, 1937-56 (2003); Eisenberg and Eisenberg Dev Biol 293, 305-15 (2006)). To test this theory, loss- and gain-of-function studies of canonical Wnt signaling were performed in a spatio-temporally restricted and cell-autonomous manner. Contrary to the prevailing view, Wnt/β-catenin signaling was required for development of cardiac progenitors in mouse embryos and in ES-derived cells, where it was necessary for even the earliest signs of cardiac gene expression in mesodermal precursors. Consistent with these findings, canonical Wnt signaling is required for induction and differentiation of cardiac progenitors in Drosophila (Zaffran and Frasch Circ Res 91, 457-69 (2002); Buckingham et al. Nat Rev Genet 6, 826-35 (2005)). In contrast, stimulation of Wnt/β-catenin signaling resulted in an enlarged heart with more proliferating cardiac cells, suggesting an instructive role in cardiac cell proliferation, likely in part through effects on cyclin D2. The efficient generation of beating cardiomyocytes and induction of cardiac gene expression from EBs grown in suspension further supports the positive role of canonical Wnt signaling in promoting cardiogenesis. Thus, it has been shown here that Wnt/β-catenin signaling is essential for heart formation in mammals and that canonical Wnt signaling can be manipulated to regulate cardiac determination and differentiation in ES cells.

FIGS. 1A and 1B Generation of tissue-specific null and stable pi-catenin. b, Western blot of ventricles from Nkx2.5-cre, ctnnb1^{tm2Kem} heterozygous, homozygous (left) and wildtype, Nkx2.5-cre, β-catenin/loxP(ex3) heterozygous (right) embryos at E12.0 using antibodies against β-catenin and GAPDH (1:50 and 1:100, respectively, Santa Cruz Biotechnology). GAPDH antibody was used as a control.

FIG. 2 Expression profiles of Brachyury. Fold change (y-axis) in cardiac gene expression is with respect to undifferentiated ES cells. Error bars indicate standard deviations.

FIGS. 3A and 3B Expression profiles of early and late cardiac genes. a, Expression levels of early cardiac genes (Nkx2.5, Tbx5, Gata4 and Mesp1) differ remarkably by Day6 in WT EBs. b, Expression of late cardiac genes (Myh6, Myh7, Mlc2a and Mlc2v) dramatically increases by day 9 in WT EBs. Fold change (y-axis) in cardiac gene expression is with respect to undifferentiated ES cells. Error bars indicate standard deviations.

FIGS. 4A-E. Canonical Wnt signaling regulates cardiac induction and differentiation in ES cells. a, Brachyury expression profiles, determined by quantitative real-time PCR (qPCR), of undifferentiated ES cells on day 0 and EBs harvested on days 3, 6, and 9 after early Wnt3a or Dkk-1 treatment compared to control. b, Beating EBs grown in suspension were counted from days 8-12. Percentage of beating EBs after early (days 4-6) or late (days 7-9) treatment with Wnt3a (left) or Dkk-1 (right). Asterisks indicate significant difference in beating percentage of treatment groups versus untreated EBs (P<0.01). c, d Immunohistochemistry of day12 EBs with anti-Islet1 (c) or anti-Tropomyosin (d). e, Cardiac gene expression after early (days 4-6, top) or late (days 7-9, bottom) treatment with Wnt3a or Dkk-1. EBs were harvested on days 3, 6 and 9 (early treatment) or days 6, 9, and 12 (late treatment). Fold change in expression of all indicated genes (y-axis) in EBs with respect to undifferentiated ES cells was assessed by qPCR. NS, not significant. *P<0.01.

### Example 2

### Identification of Genes Affected by Stabilized β-Catenin

To carry out a genome-wide search for second heart field (SHF) genes affected by stabilized (β-catenin, Rosa-YFP^{+/-}; Islet1-cre^{+/-}; β-catenin(ex3)loxP^{loxP/+} embryos were generated. These embryos showed yellow fluorescent protein (YFP) expression in SHF progenitors and their derivatives. At embryonic day (E) 9, these embryos were dissected and trimmed to exclude unnecessary cells (head and tail tissues). This embryonic stage was selected to avoid secondary gene changes that can occur after heart failure. The YFP⁺ cells were purified by fluorescence-activated cell sorting (FACS) in the Gladstone Flow Cytometry Lab (FIG. 5). Rosa-YFP-negative embryonic tissue was used as the gating control (FIG. 5, left).

FIG. 5. Histograms showing YFP+ cell populations from control (Islet1-cre, left), wildtype (Rosa-YFP; Islet1-cre, middle) and mutant (Islet1-cre; β-catenin(ex3)loxP, right) embryos at E9.5. YFP+ cells used for sorting are indicated in rectangles.

Each E9.5 embryo yielded ˜3000 YFP cells and about 10 ng of total RNA. The amount of RNA (10 ng) was enough to generate one sample for Affymetrix arrays. Three experimental replicates were used for this microarray analysis. The total RNA was amplified with the WT-Ovation Pico RNA Amplification System, fragmented and labeled with the FL-Ovation cDNA Biotin Module V2 (Nugen). Briefly, a DNA/RNA heteroduplex double-stranded cDNA were synthesized from the total RNA by RT-PCR and PCR. The cDNA was further amplified by the SPIA isothermal linear amplification. After purification, the resulting cDNA was fragmented and labeled for hybridization with Affymetrix GeneChips. The hybridization, staining and scanning of the Affymetrix GeneChips were performed in the Gladstone Genomics Core Lab. Raw data were further analyzed, and the number of potential candidates was narrowed down. Table 2 shows selected genes that are significantly dysregulated in this array.

TABLE 2

| Gene symbol | Fold change |
|---|---|
| Ndrg1 | 11 |
| Ndrl | 8.2 |
| Myct1 | 7.5 |
| Ier3 | 7.5 |
| Bhlhb2 | 6.5 |
| Fgf11 | 5.2 |
| Fgf9 | 2.6 |
| Fgf4 | 1.9 |
| Ror alpha | 4.9 |
| Sox 17 | 3.5 |
| Sox 18 | 2.2 |
| Sox 10 | 2.1 |
| Tbx20 | −1.9 |
| Isl1 | −2.3 |
| Shh | −2.4 |
| Myocd | −2.6 |
| Smyd1 | −2.6 |
| Wif1 | 3.6 |
| Apcdd1 | 3.5 |
| Dkk1 | 3.4 |
| Dkk2 | 3 |
| Dkk3 | 1.8 |
| Axin2 | 1.7 |
| T | 2.5 |
| CCng2 | 2.6 |
| Tgfbi | 2.5 |
| Tgf alpha | 2 |
| BMP2 | 2.5 |
| BMP4 | 1.8 |

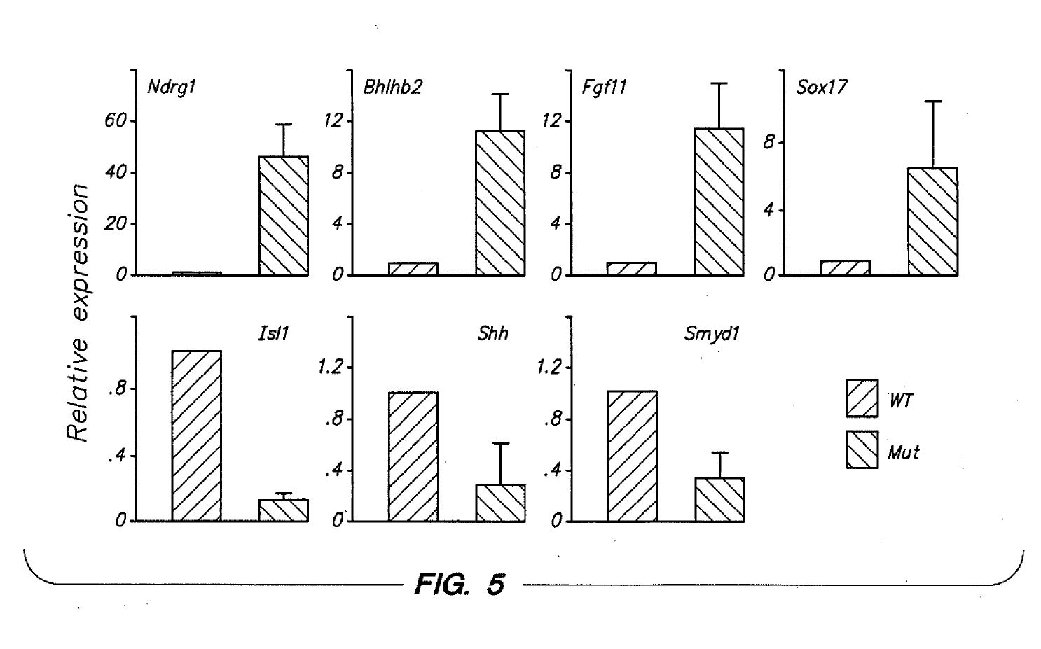

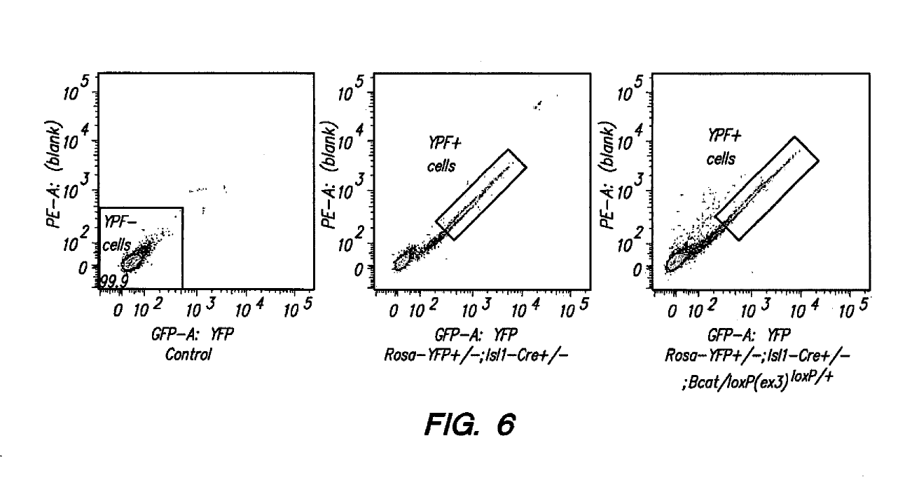

Many feedback target genes (Wnt signaling components) of canonical Wnts, such as Dks, WifI, Apcdd1, and Axin2, were upregulated in mutants, supporting the quality of the data set. Noticeably, cell-cycle-related genes, such as Ndrg1, Ndr1, and Myct1, were greatly upregulated in mutants. The N-myc downstream-regulated gene-1 (Ndrg1) encodes a novel protein involved in cellular differentiation. Kokame et al. J Biol Chem 1996; 271:29659-65; van Belzen et al. Lab Invest 1997; 77:85-92. The myc target 1 (Myct1) is a direct target of the oncogene, c-Myc, and regulates genes implicated in cancer. Cohen and Prochownik Cell Cycle 2006; 5:392-3. Consistently, genes encoding growth factors, such as Fgfs, Tgfs and BMPs, were highly upregulated. SRY-box containing genes, Sox17, 18 and 10, were also upregulated. Canonical Wnt signaling positively regulates Sox17, which is essential for cardiac specification in ES cells. Liu et al. Proc Natl Acad Sci USA 2007; 104:3859-64. In the upregulated gene set, basic helix-loop-helix domain containing, class B2 (Bhlhb2) encodes a sequence-specific transcriptional repressor, and its homologues are involved in the processes of cell cycle, differentiation, and the regulation of the circadian rhythm. Zawel et al. Proc Natl Acad Sci US A 2002; 99:2848-53; Boudjelal et al. Genes Dev 1997; 11:2052-65; Sun et al. Nat Immunol 2001; 2:1040-7; Honma et al. Nature 2002; 419:841-4. It was found that conserved Lef/Tcf binding sites in the loci of many of the upregulated genes, implying that they may be direct targets of Wnt/β-catenin signaling. A few genes appear to be significantly downregulated in the mutants. These include Isl1, Shh, Myocd, and Smyd1. Isl1 is an SHF marker and required for SHF development. Cai et al. Cell (2003) 5:877-89. Shh is required for cardiac morphogenesis and a downstream target of Islet1. Lin et al. Dev Biol 2006; 295:756-63. Therefore, the downregulation of Shh could be due to reduced Islet1 expression. However, conserved Bhlhb2 binding sites were found in the Shh locus, suggesting that Wnt/β-catenin signaling may also repress Shh expression through Bhlhb2. Myocardin (Myocd) is a transcriptional coactivator involved in myogenesis. Pipes et al. Genes Dev 2006; 20:1545-56. SET and MYND domain containing 1 (Smyd1) is a histone methyltransferase important for cardiac morphogenesis. Gottlieb et al. Nat Genet 2002; 31:25-32. Interestingly, Smyd1 knockout (KO) embryos show an enlarged heart (Gottlieb et al. (2002) supra), similar to those of embryos with the stabilized β-catenin. Kwon et al. Proc Natl Acad Sci USA 2007; 104:10894-9. Some of the candidate genes were by quantitative real-time PCR (FIG. 6).

FIG. 6. Quantitative real-time RT-PCR of indicated genes from YFP+ cells sorted from WT (Rosa-YFP; Islet1-cre, left bars) and Mut (Rosa-YFP; Islet1-cre; β-catenin(ex3)loxP, right bars) embryos. Error bars indicate standard deviations.

### Example 3

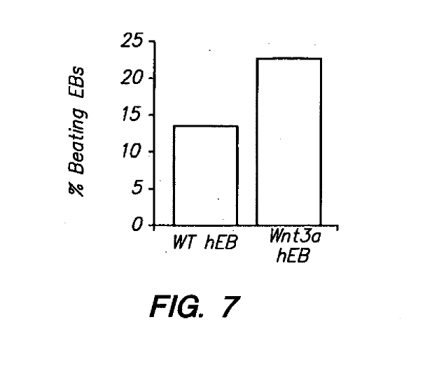

Differentiating human embryonic stem (ES) cells (H9) were treated with Wnt-3a. As shown in FIG. 7, Wnt-3a significantly increased the number of beating hES cells. Thus, canonical Wnt promotes human ES cells to differentiate into cardiomyocytes.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of inducing cardiomyogenesis in a population of stem cells or progenitor cells, the method comprising inducing a canonical Wnt signaling pathway in the stem cells or progenitor cells.

2. The method of claim 1, wherein said canonical Wnt signaling pathway is induced by contacting the stem cells with a Wnt ligand.

3. The method of claim 2, wherein the Wnt ligand is soluble Wnt3a.

4. The method of claim 1, wherein said inducing occurs before mesoderm commitment.

5. The method of claim 1, wherein at least about 10% of the stem cell population differentiates into cardiomyocytes.

6. The method of claim 1, wherein from about 10% to about 50% of the stem cell population differentiates into cardiomyocytes.

7. The method of claim 1, wherein at least about 50% of the stem cell population differentiates into cardiomyocytes.

8. A method of generating a population of cardiomyocytes from a population of stem cells or progenitor cells, the method comprising contacting the stem cells or progenitor cells with an agent that induces canonical Wnt signaling.

9. The method of claim 8, wherein the agent is a Wnt ligand.

10. The method of claim 9, wherein the Wnt ligand is soluble Wnt3a.

11. The method of claim 8, wherein said contacting is carried out in vitro.

12. The method of claim 8, wherein at least about 10% of the stem cell population differentiates into cardiomyocytes.

13. The method of claim 8, wherein from about 10% to about 50% of the stem cell population differentiates into cardiomyocytes.

14. The method of claim 8, wherein at least about 50% of the stem cell population differentiates into cardiomyocytes.

15. The method of claim 14, further comprising separating cardiomyocytes from non-cardiomyocyte cells.

16. The method of claim 15, wherein said separation comprises contacting the cells with an antibody specific for a cardiomyocyte-specific cell surface marker.

17. The method of claim 16, wherein the cardiomyocyte-specific cell surface marker is selected from troponin and tropomyosin.

18. The method of claim 8, wherein the cells are present in a matrix.

19. A method of inducing cardiomyogenesis in a population of stem cells or progenitor cells, the method comprising increasing the level of β-catenin in the stem cells.

20. The method of claim 19, the method comprising genetically modifying the stem cells or progenitor cells with an expression construct that comprises a nucleotide sequence encoding β-catenin, wherein the encoded β-catenin is produced in the stem cells or progenitor cells.

21. The method of claim 20, wherein the expression construct is a viral construct.

22. The method of claim 21, wherein the expression construct is a recombinant adeno-associated virus construct.
